(19) United States
(12) Patent Application Publication
(10) Pub. No.: US 2016/0334395 A1
(43) Pub. Date: Nov. 17, 2016

(54) HYDRATION LAYER/LIPID BILAYER STRUCTURES

(71) Applicant: NANYANG TECHNOLOGICAL UNIVERSITY, Singapore (SG)

(72) Inventors: Nam-Joon CHO, Singapore (SG); Seyed Ruhollah TABAEI AGHDA, Singapore (SG); Joshua Alexander JACKMAN, Singapore (SG)

(21) Appl. No.: 15/112,509

(22) PCT Filed: Jan. 21, 2015
(86) PCT No.: PCT/SG2015/000014

§ 371 Date: Jul. 19, 2016

Related U.S. Application Data
(60) Provisional application No. 61/929,740, filed on Jan. 21, 2014.

(51) Int. Cl. G01N 33/543; G01N 33/573; G01N 33/92

(52) U.S. Cl. CPC G01N 33/54306; G01N 33/92; G01N 33/573

(57) ABSTRACT

A method for forming a hydration layer/lipid bilayer structure on a solid support is provided. The method includes contacting a solution comprising at least one polar lipid and a water-miscible alcohol as a solvent with the solid support; and adding water to said solution at a predetermined rate, thus inducing formation of a hydration layer on the solid support surface and formation of a planar lipid bilayer on the hydration layer, wherein the hydration layer has an average thickness of at least 2 nm as well as the thus obtained solid supports with a hydration layer/lipid bilayer structure.

### CROSS REFERENCE TO RELATED APPLICATIONS

This application claims the benefit of priority of U.S. Provisional Patent Application No. 61/929,740 filed Jan. 21, 2014, the contents of which being hereby incorporated by reference in its entirety for all purposes.

### FIELD OF THE INVENTION

The present invention relates to solid-supported lipid bilayer structures and methods for their production.

### BACKGROUND OF THE INVENTION

Phospholipid membranes on solid supports offer a two-dimensional, biocompatible thin film that is useful for applications such as biofouling-resistant coatings, biosensors, and cell culture platforms. In addition, single lipid bilayers or solid-supported lipid bilayers are widely explored because they mimic the fundamental properties and architecture of biological membranes, including thickness, two-dimensional fluidity, and electrical insulation, and are in principle suitable for hosting membrane proteins (Sackmann, Science 1996, 271 (5245), 43-48). This makes them particularly suitable for, studies of biological membranes and molecules interacting therewith.

Hydrophilic solid supports offer a platform to improve the stability and lifespan of the lipid bilayer and enable characterization by surface-sensitive measurement tools. For sensing applications, the intended goal of the platform is a key determinant in choosing the type of solid support, in particular with respect to both, the material composition and nanostructure. Indeed, different solid supports provide for specific properties, such as, for example, optical transparency (e.g., glass and indium tin oxide) or high refractive index (e.g., gold and titanium oxide). The growing numbers of applications and researchers in this field have in turn spurred efforts to develop a simple and general method to form supported lipid bilayers on hydrophilic substrates with different material compositions, which is a difficult feat considering that the surface properties of individual materials vary considerably.

There has been significant interest to develop a simple and robust technique for planar bilayer fabrication. At present, there are two main techniques to produce planar bilayers that fully coat a solid support: Langmuir-Blodgett (LB) deposition and, more commonly, vesicle fusion. The latter involves vesicle adsorption and spontaneous rupture, which can occur via several mechanisms depending on the experimental conditions and vesicle characteristics (Reimhult et al. Langmuir 2006, 22, (7) 3313-3319).

A key feature of vesicle fusion is that planar bilayer formation typically occurs on a limited set of hydrophilic substrates such as borosilicate glass (Weirich et al Biophysical Journal 2010, 98, (1) 85-92) mica (Egawa et al. Langmuir 1999, 15, (5), 1660-1666) and silicon oxide (Seeger et al. J of phys Chem B 2010, 114, (27), 8926-8933). By contrast, vesicles adsorb and remain intact on gold (Keller et al. Biophysical Journal 1998, 75, (3) 1397-1402) titanium oxide and aluminum oxide (Reviakine et al. J Chem Phys 2005, 122, 204711). This limitation necessitates additional optimization of experimental conditions to promote vesicle rupture by various factors such as ionic strength (Boudard et al. Thin solid films 2006, 495, (1-2), 246-251), solution pH (Cho et al. Langmuir 2010, 26, (20), 15706-15710), osmotic shock as well as addition of divalent cations or membrane active peptides. Vesicle properties can also be optimized, including size, lipid composition, osmotic pressure and lamellarity.

In spite of all these parameters and the available possibilities to tune them, the outcome of vesicle adsorption is generally regarded as surface-specific, leading to the formation of a saturated vesicle adlayer (e.g., on gold and titanium oxide) or a supported lipid bilayer (e.g., on silicon oxide and mica). In line with this widely accepted knowledge in the field, Groves et al. (Science 1997, 275 (5300), 651-653) reported that several oxide film substrates act as barriers which prevent the formation of fluidic supported lipid bilayers: Type I barriers such as aluminum oxide prevent vesicle adsorption, while Type II barriers such as indium tin oxide and chrome support vesicle adsorption but the resulting phospholipid assemblies are effectively immobile.

Several substrates, such as aluminum oxide, are known to have appreciable surface hydration in aqueous environments that acts as a hydrophilic barrier and, hinders vesicle adsorption. The degree of surface hydration, manifesting as tightly bound water molecules at the solid-liquid interface, is believed to influence the kinetics of vesicle rupture and bilayer formation. Interfacial water on some substrates like aluminum oxide is reported (Tero, Materials 2012, 5 (12), 2658-2680) to be more tightly bound than on silicon oxide, suggesting that its influence on the corresponding lipid-substrate interaction may be even more significant. However, there has been limited attention to understand how interfacial water contributes to the role of a barrier to vesicle adsorption. Rather, fabrication efforts have mainly attempted to overcome the concomitantly weak adhesion energy by incorporating a vesicle-destabilizing agent (e.g., polyethylene glycol, AH peptide) or by chemically functionalizing the aluminum oxide surface via silianization (Lazzara et al Nano 2011, 5 (9), 6935-6944) or covalent attachment of tethered lipid anchors (Roskamp et al. 2008, 9 (13), 1920-1924).

Mager et al. (Langmuir 2008, 24 (22), 12734-12737) reported bilayer formation on aluminum oxide by using the bubble collapse deposition (BCD) method. However, this fabrication process is complex and bilayers could only form in continuous patches up to 200 μm diameter.

Cho et al. (2010 supra) reported the spontaneous formation of a supported lipid bilayer on titanium oxide by changing solution pH and thus increasing the vesicle-substrate adhesion energy. As a result, vesicles adsorb and remain intact at neutral pH conditions, because there is electrostatic repulsion, whereas vesicles rupture to form a supported lipid bilayer in acidic pH conditions due to electrostatic attraction.

Given the wide range of experimental parameters, it can be difficult to identify the right set of conditions to generate a planar bilayer. Another drawback is that vesicle fusion requires at least several hours including the time needed for vesicle preparation.

An alternative lipid bilayer fabrication technique uses solvent-exchange to bypass the requirements for vesicle preparation. In this case, lipids dispersed in alcohol are deposited on a solid support followed by solvent-exchange with an aqueous solution in order to promote a series of phase transitions with increasing water fraction that leads to the formation of a supported lipid bilayer with a single lipid. Such a method has so far only been used with a covalently tethered monolayer and/or on glass substrates—a known substrate for supported lipid bilayer fabrication via the commonly used vesicle fusion technique (Shenoy et al. 2010 RSC Soft Matter, 6, 1263-1274; Hohner et al. 2010 Biointerphases 5 (1) 1-8). As lipid self-assembly on different solid supports occurs via different mechanisms and some substrates are known to be fully resistant to lipid adsorption, it is neither known nor predictable if methods inspired by solvent-exchange can be used to form bilayers on other types of substrates. Current attempts have only used lipid solvent-exchange with phospholipids.

Several biological molecules are critical to mimicking cell membranes. A key example is sterols which represent a class of biological molecules different from the phospholipids that have been used so far. Sterols have very different self-assembly behaviors in solvent and dry systems. Indeed, phase separation of cholesterol and phospholipid before successful bilayer formation is a key challenge to existing fabrication methods

One sterol, cholesterol, is a principal component of mammalian cell membranes and is inhomogeneously distributed among various membranes of the cell. The highest concentrations of cholesterol are generally found in the plasma membranes, in which the cholesterol concentration can approach 45-50 mol % relative to other lipids (e.g., in erythrocytes). By contrast, intracellular membranes, e.g., in the endoplasmic reticulum, the Golgi apparatus, in lysosomes, and mitochondrial membranes, contain significantly less or no cholesterol. Moreover, in a variety of diseased states, cellular membranes accumulate high concentrations of cholesterol, thereby affecting normal cellular function. The formation of crystalline cholesterol domains in biological membranes at cholesterol concentrations above solubility limits can contribute to abnormal pathologies such as atherosclerosis.

A major way by which cholesterol modulates the functions of a cellular membrane is by affecting its physical properties. A wealth of previous efforts employing model membranes establish that the presence of cholesterol influences spatial distribution of membrane components by promoting domain formation within single membranes because of its differential affinity for saturated lipids and sphingomyelin. Moreover, cholesterol has a strong ordering effect on membrane phospholipids by influencing the gel to liquid-crystalline phase transition and altering membrane fluidity (or rigidity). With increasing knowledge about the non-ideal mixing of phospholipids and cholesterol, it is becoming increasingly apparent that the structural properties and phase behavior are strongly dependent on the type (or state) of the model membrane (e.g., monolayer, bilayer, multilayer film, giant vesicle).

Studies of supported membranes containing high concentrations of cholesterol are sparse presumably because of the challenges associated with preparing such membranes. The primary method available to form cholesterol-rich supported bilayers is Langmuir-type transfer processes that involve two successive transfers of lipid monolayers (Crane et al. Biiophys J. 2004, 86 (5), 2965-2979). However, it is difficult to produce equilibrium bilayer phases by this approach (Stottrup et al. Biophys J. 2004, 86 (5), 2942-2950) because of the lack of alignment between domains found in the two leaflets (Stottrup et al 2004 ibid). Alternatively, vesicle fusion, another widely used method to prepare supported bilayers, can be employed. However, the preparation of small vesicles containing high cholesterol concentrations leads to substantial heterogeneity in the compositions of individual vesicles (Huang et al. Biomembranes 1999, 1417, (1), 89-100), and different fusion rates of different subpopulations of vesicles, all of which complicate the fusion process often resulting in supported membranes, whose compositions vary significantly from the parent lipid stock (Ibarguren et al. Biomembranes 2010, 1798, (9), 1735-1738). Moreover, supported bilayers containing high cholesterol fractions are difficult to prepare (Sundh et al. Phys. Chem. Chem. Phys. 2010, 12 (2) 453-460) because cholesterol-rich vesicles have higher apparent transition temperatures and appreciably larger bending rigidities that hinder vesicle formation and rupture (in some cases (Redondo-Morata et al. Langmuir 2012, 28, (35), 12851-12860) formation of discontinuous bilayer patches is reported). As such, using the vesicle fusion method, only bilayers containing 20-30 mol % cholesterol can be reproducibly prepared. For vesicles containing higher cholesterol fractions, alternate approaches that facilitate vesicle rupture, such as by addition of a membrane-active peptide (Cho et al JACS 2007, 129, (33), 10050-1) or application of hydrodynamic force (Simonsson et al. JACS 2011, 133, (35), 14027-32), have proven successful, although these methods are rather complicated, require additional technical resources, and the corresponding mechanisms remain poorly understood.

Another drawback of known solid-supported lipid bilayers is that the properties of the bilayer are affected by interactions with the underlying substrate. These interactions may lead to an increased viscosity and lower diffusivity of the layer facing the substrate. This reduces the fluidity of the bilayer and may make it more or less immobile. Another difficulty that arises from these interactions with the solid support is that any integral membrane protein with domains that extend beyond the hydrophilic headgroups of the bilayer encounter steric hindrance from the substrate. This affects protein activity, making it difficult to interpret experimental results. The development of solid-supported lipid bilayers with greater separation distance from the solid support would improve reconstitution of functional membrane proteins, although the goal remains elusive.

In view of the above, there exists a principal need for solid-supported lipid bilayers which can mimic cellular membranes that contain high amounts of cholesterol and which are less susceptible to interactions with the solid support as well as alternative methods for forming them.

### SUMMARY OF THE INVENTION

The present invention is based on the inventors' surprising finding that the above need can be met by forming hydration layer/lipid bilayer structures on a solid support wherein the hydration layer has an average thickness of at least 2 nm. More specifically, it has been found that such thicker hydration layers minimize the interaction with the underlying solid support and provide for lipid bilayers that can accommodate high cholesterol concentrations along with other important biological molecules.

Accordingly, in a first aspect, the present invention is directed to a method for forming a hydration layer/lipid bilayer structure on a solid support, comprising the steps of: contacting a solution comprising at least one polar lipid and optionally a sterol, such as cholesterol, and a water-miscible alcohol as a solvent with the solid support; and adding water to said solution at a predetermined rate, thus inducing formation of a hydration layer on the solid support surface and formation of a planar lipid bilayer on the hydration layer, wherein the hydration layer has an average thickness of at least 2 nm.

Another aspect of the invention relates to a solid support comprising a hydration layer/-lipid bilayer structure wherein the hydration layer has an average thickness of at least 2 nm obtainable according to the method as described herein.

Other aspects of the invention will be apparent to a person skilled in the art with reference to the following drawings and description of various non-limiting embodiments.

### BRIEF DESCRIPTION OF THE DRAWINGS

The drawings are not necessarily drawn to scale, emphasis instead generally being placed upon illustrating the principles of various embodiments. In the following description, various embodiments of the invention are described with reference to the following drawings.

FIG. 1. QCM-D Monitoring of Vesicle Fusion and SALB Methods. Δf (blue) and ΔD (red) are presented. (A-B) Silicon oxide, and (C-D) Gold. SALB arrows show (1) buffer, (2) isopropanol addition, (3) lipid (0.5 mg/ml DOPC in isopropanol), and (4) buffer addition.

FIG. 2. QCM-D Measurement Responses Obtained for Supported Lipid Bilayers Formed via the SALB Procedure. (A) frequency and (B) energy dissipation signal as a function of time are presented throughout the entire process. The measurement baseline was recorded in aqueous buffer solution, followed by solvent-exchange with isopropanol (label 1), addition of 0.5 mg/mL POPC lipid in isopropanol solution (label 2), and solvent-exchange returning to aqueous buffer solution (label 3). (C) The frequency shift associated with lipid adsorption in isopropanol solution onto the solid support is reported along with the final frequency and energy dissipation values for supported lipid bilayers in aqueous solution after completion of the SALB experimental procedure. The average and standard deviation of each measurement value are reported for n=3 independent experiments per substrate.

FIG. 3. Observation of Fluidic Lipid Bilayers on Aluminum. Oxide. (A) Time-lapsed fluorescence micrographs are presented for a supported lipid bilayer on aluminum oxide formed via the SALB procedure. The dark spot in the image center corresponds to the bleached spot. (B) Normalized intensity profiles of the bleached spot on aluminum oxide before (red squares) and after recovery (blue circles). Panels (C) and (D) present similar results obtained for a supported lipid bilayer on silicon oxide formed via the SALB procedure. In Panels (A) and (B), the scale bars are 20 μm.

FIG. 4. FRAP Analysis of Supported Lipid Bilayers as a Function of Ionic Strength. Diffusion coefficient of lateral lipid mobility is presented as a function of ionic strength by varying the NaCl concentration. Reference measurements were also made for a supported lipid bilayer on silicon oxide. The average and standard deviation of each value are reported for n=5 measurements.

FIG. 5. Total Interaction Energy of Lipid-Substrate Interactions on Solid Supports. Total interaction energy as a function of separation distance between a supported lipid bilayer and aluminum oxide or silicon oxide was estimated based on extended-DLVO theory. In the model calculations, different values corresponding to the decay length of the hydration force were tested, including 0.25 nm on (A) aluminum oxide and (B) silicon oxide. Additional plots are presented for (C) 0.35 nm decay length on aluminum oxide, and (D) 0.20 nm decay length on silicon oxide. The graphs presented do not show the short-range, attractive interaction which typically occurs at separation distances below 0.3 nm. The arrows (labeled ionic strength) in each panel depict the trend in the total interaction energy with increasing ionic strength.

FIG. 6. QCM-D responses of planar bilayer formation on different surfaces using vesicle spreading and SALB deposition method. (A) Frequency (top panel) and dissipation (lower panel) shifts for the adsorption of lipid vesicles to, Al₂O₃, Cr, Tin-Oxide, TiO₂, and Au. DOPC vesicles were added at t=5 min after establishing the baseline for the frequency and dissipation in the running buffer solution (10 mM Tris, 150 mM NaCl, pH 7.5) (B) Frequency and dissipation shifts for lipid adsorption on to Al₂O₃, Cr, Tin-Oxide, TiO₂ and Au using SALB deposition method. Arrows correspond to the injection of buffer (10 mM Tris, 150 mM NaCl, pH 7.5) (1), isopropanol (2), lipid mixture (0.5 mg/ml DOPC in isopropanol) (3) and buffer (4). (C) QCM-D frequency changes upon lipid adsorption onto different substrates in isopropanol solution (corresponding to step 3 in the SALB protocol). (D) Summary of the final frequencies and dissipations corresponding to bilayer formation on the examined substrates using SALB method. The dashed-lines define the range of QCM-D Δf and ΔD expected for planar bilayer.

FIG. 7. Minimum Lipid Concentration Required for Planar Bilayer Formation by the SALB Method. Changes in QCM (A) frequency and (B) energy dissipation as functions of time are presented throughout the entire process. Panels (C) and (D), representing fragments of panel (A), show in detail the frequency shift after the moments indicated by arrows 2 and 3, respectively. A final change in frequency of −26 Hz (dotted line) corresponds to the formation of a complete planar bilayer.

FIG. 8. Stages of Planar Bilayer. Formation via SALB Method. Epifluorescence microscopy was performed to characterize lipid assemblies on silicon oxide. Depending on the bulk lipid concentration, the SALB method resulted in the formation of one of several types of lipid assemblies: (A) lipid bilayer islands at 0.05 mg·mL-1 concentration; (B) incomplete planar bilayer patches at 0.1 mg·mL-1 concentration; and (C) complete planar bilayer 0.25 mg·mL-1 concentration. FRAP experiments were performed on samples presented in panels B and C. The left and right images correspond to immediately and 1 min after photobleaching, respectively.

FIG. 9. Lipid Bilayer Islands observed by Atomic Force Microscopy. Imaging was performed to characterize the morphology of lipid layers on silicon oxide: (A) 0.05 mg·mL⁻¹ lipid, and (B-1) 0.1 mg·mL⁻¹ lipid. The measurements were recorded in aqueous buffer solution following completion of the SALB procedure. (B-2) Surface area histograms of the area detected (C) Mesoscopic lipid bilayer fragments of nonuniform shape were observed and (D) corresponding histogram analysis showed that the height was around 4-5 nm. For 0.25 mg/mL lipid concentration, (E-F) formation of a complete planar lipid bilayer was observed, as indicated by a smooth surface. (G) A 2×2 μm square segment of the lipid layer was removed via AFM tip scanning and (H) corresponding height profile analysis immediately after removal is presented.

FIG. 10. Influence of Lipid Concentration on Planar Bilayer Formation by the SALB Method. SALB experiments were performed using various organic solvents, including isopropanol, ethanol and n-propanol, as a function of lipid concentration. QCM-D monitoring was employed to track solvent-assisted lipid self-assembly. The final measurement values are reported for changes in (A) frequency and (B) energy dissipation, and (C) Adsorption of lipids in isopropanol onto silicon oxide surface. The measurement values are presented as a function of lipid concentration corresponding to adsorbed lipids in aqueous buffer solution following completion of the SALB procedure.

FIG. 11. Relative Changes in QCM-D Frequency and Energy Dissipation for SALB Experiments. (A) The QCM-D measurement responses are reported based on the final changes in frequency and dissipation for each individual SALB experiment. Each data point is replotted from FIG. 10, and represents one individual experiment performed at different lipid concentration and starting organic solvent. Isopropanol: circles, N-propanol: triangles, Ethanol: squares. (B) Proposed Mechanism of SALB formation Process. Lipid adsorption first occurs in organic solvent (not shown). The organic solution containing lipids is then replaced by aqueous buffer solution. On the surface, the experimental data shows that the aqueous buffer solution promotes the formation of bilayer islands. In the solution, the aqueous buffer solution mixes with the organic solvent during the exchange process and likely induces lipids to form monomers and micelles, which are typically found in solutions with intermediate water fraction. Depending on the island density on the surface, different types of lipid structures may form on the substrate due to the interaction between bilayer islands on the surface and monomers/micelles in the solution: (I) Bilayer, if there is low to intermediate island density, then lipids in solution can fuse with edges of a bilayer island and propagate bilayer expansion. In this case, the islands serve as two-dimensional nucleation sites. (II) Wormlike micelle formation, if there is high island density, then lipids in solution can fuse with the bilayer islands. Owing to the high density of islands, three-dimensional nucleation occurs and lipids not only fuse to form a two-dimensional, planar bilayer but can also form filamentous structures that project outwards from the surface. (III) Vesicle, this case is limited to high lipid concentrations in ethanol. During the period of mixing between aqueous buffer and lipids in ethanol, the solvent properties induce vesicle formation and vesicle adsorption onto the surface causes the characteristic QCM-D responses.

FIG. 12. Effect of Organic Solvent on SALB Method. Final values are reported for (A) Δf and (B) ΔD. The same experimental procedure was repeated for all solvents.

FIG. 13. FRAP Measurement of SALB Bilayers Formed in Different Organic Solvents. False color fluorescence images (100×100 μm) of DOPC bilayers doped with 0.5% Rho-PE. (A) Right and left images are immediately and 60 sec after photobleaching, respectively. (B) Normalized intensity profiles show recovery. (C) Diffusion coefficients for Rho-PE lipid.

FIG. 14. Observation of Fluidic Cholesterol-Enriched Supported Membranes on Glass. (A-E) Fluorescence micrographs were recorded for supported lipid bilayers formed on a glass substrate. The precursor mixture in isopropanol solution had a molar ratio of (100−x) mol % DOPC lipid and x mol % ChoI, and contained 0.5 wt % fluorescent Rhodamine-PE lipid; x ranged from 0 to 50 mol %. Images were recorded immediately (top) and 1 min (middle) after photobleaching. The dark spot in the image center corresponds to the photobleached region. The scale bars are 20 μm. Surface area histograms of individual dye-excluded domains within each sample are also presented (bottom).

FIG. 15. Characterization of Dye-Excluded Domains by Tapping Mode Atomic Force Microscopy. (A) AFM height mode image of a SALB-formed supported lipid bilayer. The composition of the precursor mixture was 70 mol % DOPC lipid and 30 mol % cholesterol. The scan size was 50 μm×50 μm. Two line scans are denoted by labels 1 and 2, respectively, and the corresponding height profiles are presented below the AFM images. (B) AFM height mode image of a representative dye-excluded domain. The scan size was 10 μm×10 μm. Two line scans are denoted by labels 1 and 2, respectively. The “1” line scan was performed to determine the height difference between the phospholipid-rich phase and dye-excluded domain. The “2” line scan was performed to determine the thickness of the phospholipid-rich phase. (C) AFM height mode image of a lipid bilayer defect created by treatment with 1 mM MβCD. The image was recorded post-rinse 30 min after MβCD application, and the scan size was 5 μm×5 μm. Two line scans are denoted by labels 1 and 2, respectively, and the corresponding height profiles are presented.

FIG. 16. Cholesterol Depletion by Methyl-β-Cyclodextrin Treatment. Fluorescence micrograph of a cholesterol-enriched supported membrane (A) before and (B) after treatment with 1 mM MβCD. The precursor mixture was 50 mol % DOPC and 50 mol % ChoI, and contained 0.5 wt % Rhodamine-PE. (C) Time-lapsed fluorescence microscopy imaging was performed in order to measure the effects of 1 mM MβCD treatment on the lipid bilayer region in panels A and B. The total surface area of the bilayer region (green color) and average fluorescence intensity (FI) of pixels in those regions are presented as a function of time upon MβCD injection (see arrow). (D-E) FRAP analysis on a representative MβCD-treated bilayer is presented. The precursor mixture was 49.5 mol % DOPC, 0.5 wt % Rhodamine-PE, and 50 mol % cholesterol. In panel D, photobleaching was performed at t=0 min. In panel E, recovery of the fluorescence intensity in the fluid phase regions is observed by t=1 min.

FIG. 17. Fluorescence Recovery Photobleaching (FRAP) Analysis of Lateral Lipid Diffusion in Cholesterol-Enriched Supported Membranes. The diffusion coefficient (in units μm²·s⁻¹) of lateral lipid mobility is reported for supported membranes with varying DOPC:ChoI molar ratios (n=10 measurements). FRAP measurements were performed before and after 1 mM MβCD treatment. Supported lipid bilayers on glass were formed by either the SALB or vesicle fusion method.

FIG. 18. Quantitative Determination of Cholesterol Fraction in Supported Membranes. (A) The amount of cholesterol incorporated within supported DOPC:ChoI lipid bilayers on silicon oxide was determined by QCM-D measurement. First, the SALB procedure was performed to form supported lipid bilayers with varying mole fractions of cholesterol in the precursor mixture (between 0 and 50 mol %). After bilayer formation, the QCM-D frequency shift was then normalized with ΔFrequency=0 Hz corresponding to the supported lipid bilayer in each experiment. 1 mM MβCD was next added (see arrow) in order to remove cholesterol from the bilayer, and the observed positive frequency shift was due to cholesterol removal from the bilayer. (B) Mole percent of cholesterol depleted from the bilayers prepared by the SALB method as a function of cholesterol fraction in the precursor mixture. For comparison, identical experiments were performed using supported lipid bilayers prepared by the vesicle fusion method.

FIG. 19. Quantitative Determination of Cholesterol Fraction in Supported Membranes. The amount of cholesterol incorporated within supported DOPC:ChoI lipid bilayers was determined by QCM-D measurement via methyl-fl-cyclodextrin (MβCD) extraction of ChoI from the bilayer. Measurements were performed for supported bilayers formed by (A) vesicle fusion on silicon oxide and (B) the SALB procedure on gold. After bilayer formation, the QCM-D frequency shift was then normalized with ΔFrequency=0 Hz corresponding to the supported lipid bilayer in each experiment. 1 mM MβCD was next added (see arrow) in order to extract ChoI from the bilayer, and the observed positive frequency shift was due to ChoI removal from the bilayer.

FIG. 20. QCM-D Analysis of AH Peptide-Mediated Planar Bilayer Formation from Cholesterol-Rich Vesicles. Using QCM-D tracking, changes in (A) frequency (ΔFn=3/3, blue) and (B) dissipation (AD, red) were measured as functions of time for vesicle adsorption onto silicon oxide. DOPC lipid vesicles containing various molar fractions of cholesterol (between 30-50 mol %) were added at t=5 min (see arrow 1) after establishing the measurement baselines. The dashed lines represent typical bilayer ranges of frequency shifts, Δf=−25 Hz, and dissipation shifts, ΔD less than 1×10⁻⁶. Vesicles containing more than 20 mol % ChoI did not rupture and remained intact, and AH peptide was added to induce vesicle rupture (see arrow 2). After bilayer formation, 1 mM methyl-β-cyclodextrin was added in order to extract cholesterol from the bilayer (see arrow 3), although minimal effects were observed. For all experiments, measurement baselines were recorded in the same aqueous buffer solution (10 mM Tris [pH 7.5] with 150 mM NaCl).

FIG. 21. QCM-D Monitoring of the Formation of Phosphoinositol (PI) containing Bilayer Using the Methods. QCM-D frequency shift (Δf, top panel) and dissipation (ΔD, bottom panel) for the third overtone were measured as a function of time during formation of PI (A) and PI(4,5)P2 (B) containing bilayer, on silicon dioxide. Lipid mixture [0.5 mg/ml of DOPC in isopropanol with (grey curves) and without 10% PI (black curves)] was added at t=5 min after establishing the baseline for the frequency and dissipation changes. Next buffer was injected leading to a final Δf and ΔD of −26±1 Hz and 0.3±0.2×10⁻⁶, respectively which corresponds to a planar bilayer. Subsequently anti-PI4P and anti-PI(4,5)P2 (5 μg/ml) was injected leading to further decrease in Δf of PI containing bilayers. No change in the Δf of pure DOPC bilayers were observed indicating specific binding of antibodies to the PI lipids.

FIG. 22. QCM-D Characterization of Kinase Activity. PI containing bilayer was formed at room temperature (23° C.) by the method and incubated with (A) PI4KIIIα and (B) PI4KIIIβ at 30° C. Finally anti-PI4P was injected to verify enzymatic conversion of PI to PI4P. No binding was observed for control bilayers.

FIG. 23. In situ Antibody Binding to PI4P Generated by Lipid Kinase in the Presence of Inhibitors. The procedure was performed to form DOPC supported lipid bilayers with 10% PI in the precursor mixture. After bilayer formation, it was treated with PI4KIIIα and β which were pre incubated with inhibitors for 30 min at room temperature. The initial baseline values are the normalized frequency shifts for the bilayers (defined as Δf Bilayer=0). The observed negative frequency shift was due to antibody binding to the bilayer. The ΔF upon antibody binding is presented as a function of inhibitor concentration. For comparison, control experiments were performed in which no inhibitor was added.

### DETAILED DESCRIPTION

Herein, the inventors demonstrate a method using solvent-assisted lipid bilayer (SALB) formation to fabricate planar bilayers on solid supports that were formerly found to be intractable substrates. Compared to previous strategies, the SALB method does not require vesicles and can be performed using a one-step procedure in less than thirty minutes. In addition, it has surprisingly been found that this method has the advantage of forming bilayers with a thicker hydration layer between the solid support and the bilayer. A thicker hydration layer separating the lipid bilayer from the support surface provides for bilayers that are less susceptible to influences from the solid support material that have previously been found to impair bilayer fluidity and the mimicking of biological membranes.

The developed method described herein is suited to form a hydration layer/lipid bilayer structure on a solid support with the hydration layer having an average thickness of at least 2 nm and comprises the steps of contacting a solution comprising at least one polar lipid and a water-miscible alcohol as a solvent with the solid support; and adding water to said solution at a predetermined rate. The addition of water induces formation of the hydration layer on the solid support surface as well as formation of a planar lipid bilayer on the hydration layer.

In various embodiments, the hydration layer has a thickness between 2 nm and 4 nm, preferably between 3 nm and 4 nm.

The term ‘lipid bilayer’, as used herein, refers to two layers of polar lipids aligned planar on the hydration layer of a solid support. In said bilayer structures, the hydrophilic headgroups are oriented such that they form the outer surfaces of the bilayer structure and are exposed to the surrounding medium, while the hydrophobic tail groups are oriented inwardly.

As used herein, the term ‘hydration layer’ refers to a thin layer of water on a given support surface. Such a hydration layer typically forms a strong short range, typically repulsive, force between polar surfaces.

As used herein the term ‘polar lipid’ refers to any suitable lipids that have polar properties in that they have a polar or even charged headgroup. The term, as used herein, includes well-known lipids, such as, for example, phospholipids, in particular glycerophospholipids, such as phosphatidylcholine, phosphatidylethanolamine, phosphatidic acid, phosphatidylinositol and/or phosphatidylserin, or sphingolipids. While these are preferred lipids, any other polar lipid known to be suitable to form lipid bilayers may similarly be used either individually or in combination, for example in combination with the afore-mentioned phospholipids.

In various embodiments the at least one polar lipid is selected from the group consisting of phospholipids, sphingolipids, fatty acids, derivatives thereof and combinations thereof. In various embodiments the at least one polar lipid comprises at least one phosphoglyceride, preferably a phosphatidylcholine.

Sphingolipids that may be used in accordance with the present invention include sphingomyelins. These may have phosphocholine or phosphoethanolamine head groups.

Fatty acids that may be used in accordance with the present invention include, but are not limited to, unsaturated or saturated C₁₀₋₂₄ fatty acids, in particular lauric acid, myristic acid, palmitic acid, stearic acid, arachidic acid, behenic acid, oleic acid, linoleic acid, alpha linolenic acid, and arachidonic acid.

In the phospholipids or sphingolipids used in accordance of the invention, the fatty acid residues are preferably selected from myristic acid, palmitic acid, stearic acid and oleic acid, more preferably from palmitic acid, stearic acid and oleic acid.

As used herein, the term ‘water-miscible alcohol’ refers to alcohols that may be mono- or multifunctional, that are freely miscible with water, i.e. do not form a separate organic phase at any concentration ratio. Examples of water-miscible alcohols include, without limitation, methanol, ethanol, and propanols, such as isopropanol and n-propanol. In various embodiments the alcohol is selected from isopropanol, n-propanol and methanol. In various embodiments the alcohol comprises or consists of isopropanol.

In various embodiments the solid support is formed from a material that has a Hamaker constant in water of at least 3×10⁻²⁰ J. The Hamaker constant in water can be taken from or determined according to Bergstrom (1997, Advances in Colloid and Interface Science, 70, 125-169).

Such materials that have a Hamaker constant in water of at least 3×10⁻²⁰ J are notoriously difficult to form lipid bilayers thereon, as they generally have hydration forces that are too repulsive for vesicle absorption/disruption formation of lipid bilayers. It was surprisingly found by the inventors that the hydration layer formed on a material that has a Hamaker constant in water of at least 3×10⁻²⁰ J are much thicker than those formed on other materials, the thickness typically ranging from about 2 nm to about 4 nm. While this is problematic for lipid bilayer formation via the vesicle absorption/disruption Method, as it inhibits bilayer formation, it has been found to rather represent an advantage when using the described method. This is due to the finding that using the described methods a planar lipid bilayer can be formed on the thick hydration layer of such a material that has a Hamaker constant in water of at least 3×10⁻²⁰ J, with the resulting thick hydration layer between the support and the bilayer allowing more precise experiments that require mimicking of biological membranes, as the influence of the support on membrane properties is minimized and membrane protein with domains that extend beyond the hydrophilic headgroups of the bilayer can be incorporated into the membrane without spatial restraints due to the proximity of the underlying support in other known systems.

Examples of materials that have a Hamaker constant in water of at least 3×10⁻²⁰ J and that can support the hydration layer/lipid bilayer structure described herein include silver (Ag), gold (Au), aluminum oxide (Al₂O₃), Barium titanate (BaTiO₃), beryllium oxide (BeO), diamond (C), cadmium sulfide (CdS), copper (Cu), magnesium aluminate (MgAl₂O₄), magnesium oxide (MgO), lead sulfide (PbS), silicon carbide (SiC), silicon nitride (Si₃N₄), strontium titanate (SrTiO₃), titanium dioxide (TiO₂), yttrium oxide (Y₂O₃), zinc sulfide (ZnS), zirconium oxide (ZrO₂), chromium (Cr), and tin oxide (SnO).

In various embodiments the solid support is selected from silver, gold, aluminum oxide, Barium titanate, beryllium oxide, diamond, cadmium sulfide, copper, magnesium aluminate, magnesium oxide, lead sulfide, silicon carbide, silicon nitride, strontium titanate, titanium dioxide, yttrium oxide, zinc sulfide, zirconium oxide, chromium, and tin oxide. In some more specific embodiments, the solid support is selected from gold, titanium dioxide, aluminum oxide, chromium, and tin oxide, preferably from gold and aluminum oxide.

In various embodiments the solution comprises the polar lipid at a concentration of about 0.1 to about 0.75 mg/ml, preferably about 0.1 to about 0.5 mg/ml.

“About”, as used herein in relation to a numerical value, refers to said numerical value±10%, preferably ±5%. For example, “about 0.1 mg/ml” therefore includes a range from 0.09 to 0.11 mg/ml.

It has been surprisingly found that the method described herein, unlike other methods of forming bilayers, usually has an upper limit for a concentration of polar lipids in that above the upper limit, no longer a lipid bilayer but other structures are formed. Accordingly, using the method described herein, a lipid bilayer will form within the lipid concentration range given above.

The lipid composition used for bilayer formation is not particularly limited and may comprise a single lipid or various different lipids.

In various embodiments, the at least one polar lipid comprises at least two different polar lipids. In preferred embodiments, the solution further comprises in addition to the at least one polar lipid, another (non-polar) lipid or lipid-like component. This additional lipid or lipid-like component may be selected from the group consisting of triacylglycerides and isoprenoids. In the triacylglycerides, the fatty acids may be selected from those listed above. Preferred isoprenoids include all steroids, particularly sterols known as biological membrane components, and more preferably include, without limitation, cholesterol, ergosterol, hopanol and/or phytosterol. The most preferred sterol is cholesterol. Also contemplated are combinations of different sterols.

In various embodiments, the solution comprises up to 50% by weight sterols relative to the total lipid content, preferably cholesterol. These high sterol contents may result in lipid bilayer structures that have a sterol content of up to 50 mol-% relative to the total lipid content.

It has been demonstrated that using the described method cholesterol-enriched bilayers can be formed, with their lipid composition reflecting the starting precursor compositions. Importantly, the method described herein allows the formation of bilayers that can incorporate sterols, such as cholesterol, at concentrations that are more than 5 times higher than those achievable with conventional techniques for supported bilayer formation (e.g., vesicle fusion).

In various embodiments the lipid bilayer therefore comprises at least 20 mol-% sterols, at least 30 mol-% sterols, and at least 40 mol-% sterols. The mole fractions of sterols in the formed bilayer reflect the starting precursor compositions. Accordingly, the solution may comprise up to 20% by weight, up to 30% by weight, up to 40% by weight up to 50% by weight, or up to 60% by weight sterols relative to the total lipid content.

In all the above embodiments, the sterols preferably include cholesterol. In various embodiments, cholesterol is the only sterol used.

In various embodiments, the solution further comprises peptides or proteins that can associate with or insert into the formed lipid bilayer. The proteins may be integral or peripheric membrane proteins, with the former including transmembrane proteins. The proteins can interact with the formed lipid bilayer by specialized protein structures, such as transmembrane domains or other structural motifs that can interact with or insert into the lipid bilayer, or by modifications, in particular lipid modifications, such as farnesylation or geranylgeranylation. In various embodiments, the proteins are naturally occurring proteins or fragments or variants thereof that are to associate with the formed lipid bilayer, for example for the characterization of their properties and functionality. In various embodiments, the protein is a phosphatidylinositol-related protein or enzyme, preferably a phosphatidylinositol-4,5-bisphosphate-3-kinase (PI3K).

Typically, these enzymes are very hard to study because phosphatidylinositols are difficult to incorporate in lipid bilayers. They are widely involved in biological signaling pathways and carry a high negative net electrical charge. Accordingly, vesicles or membranes containing phosphatidylinositols have a strongly negative surface charge that hinders lipid-substrate interactions due to high electrostatic repulsion. Hence, it is very difficult to form supported lipid bilayers containing phosphatidylinositols by conventional fabrication techniques and those formed have poor quality, which is insufficient for applications testing.

In various embodiments the alcohol solution is essentially non-aqueous. “Essentially non-aqueous”, as used in this context, means that in various embodiments the solution before the step of adding the water contains less than 40% by weight, preferably less than 25% by weight, more preferably less than 10% by weight, most preferably less than 2% by weight water.

In various embodiments the water is added in form of an aqueous solution, preferably a buffered aqueous solution.

In various embodiments the buffered aqueous solution may include salts such as sodium chloride, calcium carbonate or any other such salt and/or buffer compounds such as but not limited to N,N-bis(2-hydroxyethyl)glycine, N-tris(hydroxymethyl)methylglycine, dimethylarsinic acid, sodium citrate, 3-{[tris(hydroxymethyl)methyl]amino}propanesulfonic acid, tris(hydroxymethyl)methylamine, 3-[N-Tris(hydroxymethyl)methylamino]-2-hydroxypropanesulfonic acid, 4-2-hydroxyethyl-1-piperazineethanesulfonic acid, 2-{[tris(hydroxymethyl)methyl]amino}ethanesulfonic acid, 2(R)-2-(methylamino)succinic acid, 3-(N-morpholino)propanesulfonic acid, piperazine-N,N′-bis(2-ethanesulfonic acid), 2-(N-morpholino)ethanesulfonic acid or any other suitable buffer compound. While the buffers may in general have pH values in the range of 2 to 11, it is preferred that the buffer compound used is suited to maintain the pH of the aqueous solution is within a range of 3-9 more preferably within a physiological range. Preferably, in various embodiments, the pH of the aqueous solution is in the range of between 7.0 and 7.8, preferably about 7.4.

Another aspect of the invention relates to a solid support comprising a hydration layer/lipid bilayer structure wherein the hydration layer has an average thickness of at least 2 nm, wherein said solid support is obtainable according to the method as described herein. Other known methods of forming lipid bilayers on intractable solid supports provide for a thinner hydration layer between the solid support and a bilayer and may even require tuning the hydration force such that the resulting hydration layer is thinner than 2 nm. In contrast to these existing methods, the method described herein results in a hydration layer/lipid bilayer structure wherein the hydration layer has an average thickness of at least 2 nm. As described above, the increased hydration layer thickness provides for numerous advantages of the obtained structures, including provision of a more suitable environment for hosting transmembrane proteins.

In the solid supports of the invention, the lipids used for bilayer formation and making up the final bilayer structure are identical to those described above in connection with the inventive methods. Similarly, the solid support materials used are the same that have been described above in relation to the methods of the invention.

As also described above, in various embodiments the lipid bilayer comprises sterols, preferably cholesterol, in particular at concentrations of up to 20, 30, 40, 50, or 60 mol-%.

By “comprising” it is meant including, but not limited to, whatever follows the word “comprising”. Thus, use of the term “comprising” indicates that the listed elements are required or mandatory, but that other elements are optional and may or may not be present.

By “consisting of” is meant including, and limited to, whatever follows the phrase “consisting of”. Thus, the phrase “consisting of” indicates that the listed elements are required or mandatory, and that no other elements may be present.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples.

### EXAMPLES

### Example 1

### Formation of a Hydration Layer/Lipid Bilayer on Oxidized Gold

Lipid Preparation.

Zwitterionic lipid, 1,2-dioleoyl-sn-glycero-3-phosphocholine (DOPC), and fluorescently labeled lipid, 1,2-dioleoyl-sn-glycero-3-phosphoethanolamine-N-(lissamine rhodamine B sulfonyl) (ammonium salt) were purchased from Avanti Polar Lipids (Alabaster, Ala.). Extruded vesicles were prepared using 50 nm pore, polycarbonate membranes and 10 mM Tris buffer (pH 7.5) with 150 mM NaCl, as previously described. For SALB experiments, dried lipids were dissolved in the appropriate solvent at 10 mg/ml lipid and diluted before experiment.

Quartz Crystal Microbalance-Dissipation (QCM-D).

A Q-Sense E4 (Q-Sense AB, Gothenburg, Sweden) instrument was used to monitor the lipid deposition process in real time. Changes in the resonance frequency and energy dissipation of a 5 MHz AT-cut piezoelectric quartz crystal were captured at the 3rd, 5th, 7th, 9th and 11th overtones, respectively. All measurements were done under flow-through conditions at a flow rate of 5.0 μL/min, by using a Reglo Digital peristaltic pump (Ismatec, Glattbrugg, Switzerland). The experimental temperature was fixed at 24.0±0.5° C. All surfaces were treated with oxygen plasma at 180 W for 1 min (March Plasmod Plasma Etcher, March Instruments, California) immediately before use.

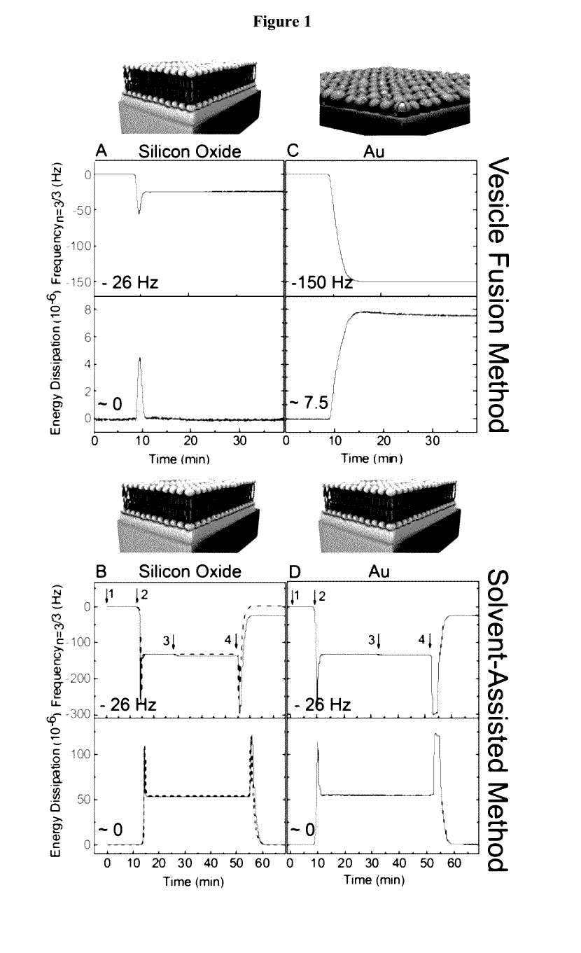

Planar bilayer formation on gold has been difficult to achieve. On gold, vesicles typically adsorb and do not rupture. As demonstrated in FIG. 1C, vesicle addition onto the gold surface led to the formation of a layer of intact vesicles (Δf and ΔD of −150 Hz and 7.5×10⁻⁶, respectively). At present, the only method available to form planar bilayers on gold is to add a specific amphipathic, α-helical (AH) peptide that is known to induce vesicle rupture (Cho et al. JACS 2007, 129, (33), 10050-10052). Based on the aforementioned experimental results on silicon oxide, the SALB method has several potential advantages to form bilayers on gold. It does not require vesicles and the bilayer properties appear to be equivalent independent of whether vesicle fusion or SALB was employed.

Strikingly, in contrast to the results observed with vesicle fusion, planar bilayer formation was observed on gold by using the SALB method (Of and AD of −26 Hz and 0.3×10⁻⁶, respectively). (FIG. 1D). Lipid adsorption in isopropanol prior to bilayer formation was also observed. As gold is intractable to spontaneous vesicle rupture, this finding supports that adsorbed lipids in isopropanol undergo a structural transformation to directly form a planar bilayer on the substrate. Compared to existing bilayer fabrication strategies on solid supports in general and gold in particular, the SALB method is much simpler and takes advantage of how lipids self-assemble in different solvents.

### Example 2

### Formation of a Lipid Bilayer on Silicon Oxide

For vesicle fusion, characteristic two-step adsorption kinetics were observed (FIG. 1A) as is known in the art. The first step involved vesicle adsorption until reaching a critical coverage, as denoted by maximum changes in the QCM-D measurement signals (Δf and ΔD of 60 Hz and 4×10⁻⁶, respectively). The critical coverage denotes the point whereupon the combination of vesicle-substrate and vesicle-vesicle interactions becomes sufficient to cause vesicle rupture. The following step involved vesicle rupture, including the release of coupled solvent from inside the vesicles. This caused a decrease in adsorbed mass and final changes consistent with a planar bilayer 10 (Δf and ΔD of −26 Hz and 0.1×10⁻⁶, respectively).

SALB experiments were conducted on silicon oxide with QCM-D monitoring (FIG. 1B). Once the SALB procedure was finished as described above, the values were consistent with previous QCM-D responses for planar lipid bilayer formation on silicon oxide 10 (Δf and ΔD of −26 Hz and 0.2×10⁻⁶, respectively). Interestingly, lipid deposition in organic solvent was observed (Δf and ΔD of −5.2 Hz and 0.1×10⁻⁶, respectively). It is currently understood that lipids form inverted hexagonal micelles in isopropanol, which suggests the structural transformation of these structures into a planar bilayer. The black, dotted curves refer to a control experiment that was performed using an identical procedure, albeit without lipid. At step 3, there was no change in measurement signal for the control experiment, further supporting that lipid adsorption in organic solvent occurs. Moreover, the final measurement values in the control experiment were equivalent to the baseline. The control experiment supports that no lipid mass was adsorbed onto the substrate and that the final changes in measurement values associated with bilayer formation upon stabilization at each step are due to lipid adsorption. Overall, the findings support that, on silicon oxide, planar lipid bilayers can be formed by using both methods.

The Hydration layer of the silicon oxide is calculated to be about 1 nm and that of gold is calculated to be 2.5 nm. The larger hydration layer formed on gold is able to support experiments involving incorporated membrane proteins as described below. Using this solvent-assisted lipid bilayer (SALB) approach, the formation of a supported lipid bilayer was formed on gold. Interestingly, the hydration mass for a supported lipid bilayer on gold was measured to be greater than for a bilayer on silicon oxide, which suggests that surface hydration inhibits vesicle rupture on gold. Similar reasoning has been offered to explain the case of titanium oxide, and supported by extended-DLVO model calculations which indicate that the hydration force is a governing parameter to influence the lipid-substrate interaction on titanium oxide.

### Example 3

### Formation of a Hydration Layer/Lipid Bilayer on Aluminum Oxide

SALB formation method is based on solvent-assisted lipid self-assembly when lipids in alcohol are deposited on a substrate and then solvent-exchange is performed leading to an increase in the water fraction. As the water fraction increases, lipids attached to the surface and in solution undergo a series of phase transitions eventually forming a supported lipid bilayer. Notably, the adhesion energy needed to stabilize a supported lipid bilayer on aluminum oxide is likely much less than that required for adsorbed vesicles to rupture, and the SALB procedure therefore offers a solution to bypass the high contact energy requirements of vesicle rupture.

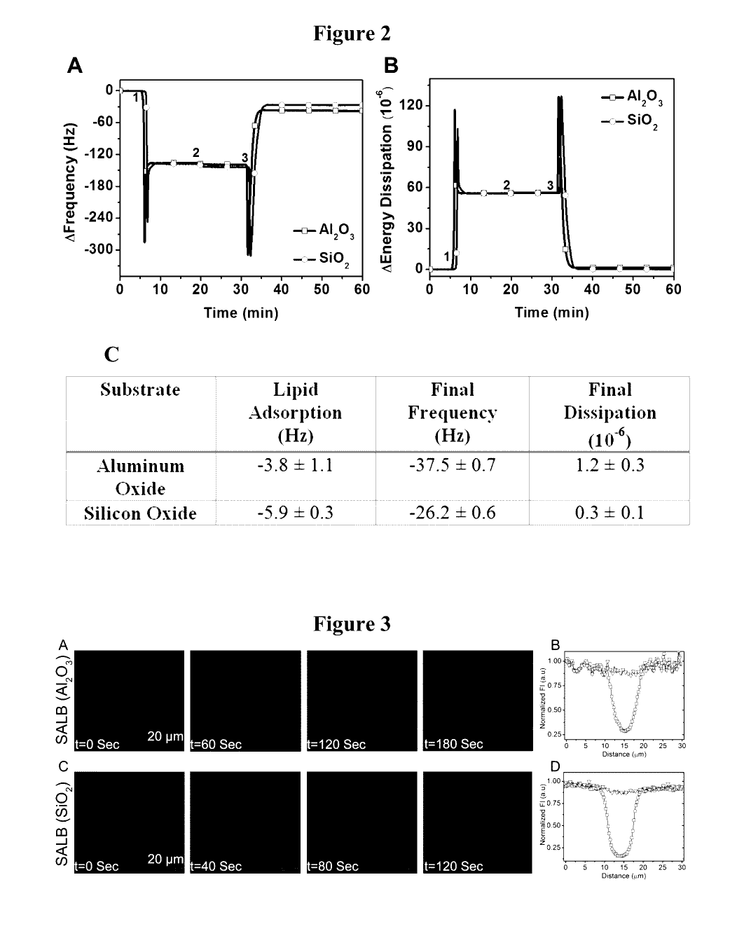

Hence, a supported lipid bilayer was formed on Aluminum oxide at pH 7.5 via the SALB procedure. FIG. 2A-B presents representative QCM-D measurement traces for SALB experiments on aluminum oxide. Similar experiments were also performed on silicon oxide for comparison. A measurement baseline was first recorded in aqueous buffer solution (10 mM Tris buffer [pH 7.5] with 150 mM NaCl), followed by exchange with isopropanol solution at t=5 min. Afterwards, 0.5 mg/mL POPC lipid in isopropanol was deposited onto the substrate at t=20 min, and lipid attachment reached equilibrium. On aluminum oxide, lipid attachment in isopropanol corresponded to a −3.8±1.1 Hz frequency shift. By contrast, on silicon oxide, lipid attachment resulted in a −5.9±0.3 Hz frequency shift which indicates a greater amount of attached lipid to silicon oxide.

After lipid attachment in isopropanol, a solvent-exchange step was performed at t=30 min in order to reintroduce aqueous solution (without lipid) and complete the SALB procedure. The final frequency and dissipation shifts are reported in FIG. 2C. On aluminum oxide, the final frequency and energy dissipation shifts were −37.5±0.7 Hz and 1.2±0.3×10⁻⁶, respectively. By contrast, on silicon oxide, the final frequency and energy dissipation changes were −26.2±0.6 Hz and 0.3±0.1×10⁻⁶, respectively, which are in agreement with, past measurements. In order to determine if the lipid layer on aluminum oxide was globally homogenous, the nonspecific adsorption of bovine serum albumin was found to be negligible and conclude that the lipid layers on aluminum oxide are greater than 97% complete.

One possible explanation for the difference in measurement values on the two substrates is a thicker hydration layer on aluminum oxide. Assuming that the difference in the frequency shifts between the aluminum oxide and silicon oxide cases is due to coupled solvent in the hydration layer and that the density of this solvent is equivalent to liquid water (1 g/cm³). By applying the Sauerbrey relationship, it was estimated that a 1 Hi shift deviation is due to a 0.177 nm thicker hydration layer. The hydration layer for a supported lipid bilayer on aluminum oxide would be ˜2.1 nm thicker (˜12 Hz shift difference) than on silicon oxide. This estimated range is consistent with previous atomic force microscopy (AFM) thickness measurements for supported lipid bilayers on aluminum oxide (˜6.5 nm) and silicon oxide (˜4.6 nm) with similar chain-length lipids under nearly equivalent pH conditions. Hence, the QCM-D measurement values indicate that a supported lipid bilayer is formed on aluminum oxide by using the SALB approach.

### FRAP Analysis of Supported Lipid Bilayer on Aluminum Oxide

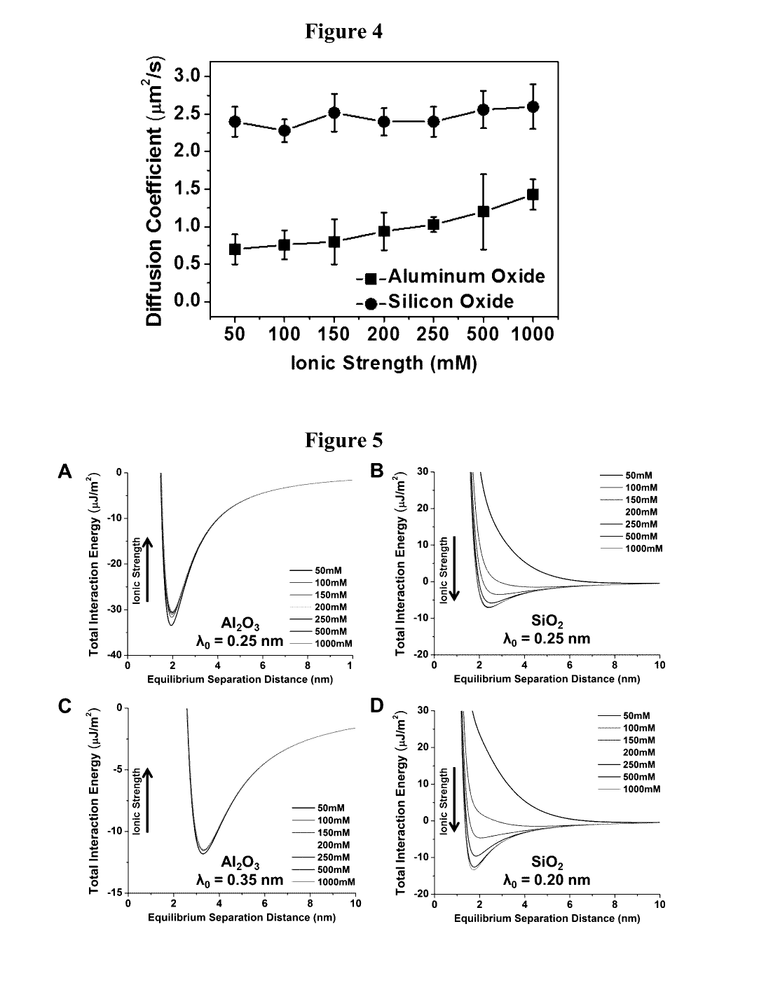

To further investigate the properties of supported lipid bilayers formed by the SALB procedure, fluorescence recovery after photobleaching (FRAP) measurements were determined. For these experiments, 0.5 wt % fluorescently labeled Rhodamine-DOPE lipid was included in the precursor mixture in isopropanol in order to visualize the supported lipid bilayers. After completion of the SALB procedure, the ionic strength of the solution was varied by titration and FRAP measurements were recorded at each ionic strength condition. Lateral lipid diffusion was observed for the supported lipid bilayer on aluminum oxide, as indicated in representative FRAP snapshots presented in FIG. 3A. At 100 mM ionic strength, the diffusion coefficient of the supported lipid bilayer on aluminum oxide was 0.76±0.19 μm²/s, which is in agreement with previous diffusion coefficients (0.62±0.21 μm²/s) obtained for lipid bilayer patches on aluminum oxide formed by the BCD method under nearly identical ionic strength and pH conditions. FIG. 3B presents normalized fluorescence intensity traces of the bleached spot and the mobile fraction was ˜86%, which is also in agreement with previous measurements. The calculated diffusion coefficient and mobile fraction are lower those that typically obtained on silicon oxide, and this difference has been attributed to stronger hydrodynamic coupling. Previous NMR studies on oxide nanoparticles also indicate that water near an aluminum oxide surface is less mobile than that near a silicon oxide surface. By contrast, at 100 mM ionic strength, the diffusion coefficient and mobile fraction of the supported lipid bilayer on silicon oxide are 2.28±0.15 μm²/s and >90%, which are in agreement with literature values. On both substrates, the FRAP measurement results are also reported as a function of ionic strength by varying the NaCl concentration (FIG. 4). For aluminum oxide, the diffusion coefficient increased with increasing ionic strength along with the mobile fraction (>90%). At 50 mM NaCl, the diffusion coefficient was 0.70±0.20 μm²/s, whereas it increased to 1.43±0.20 μm²/s at 1000 mM NaCl. Hence, depending on the ionic strength, the diffusion coefficient varied by up to two-fold for a supported lipid bilayer on aluminum oxide. By contrast, on silicon oxide, the diffusion coefficient of a supported lipid bilayer was largely independent of the ionic strength, maintaining a value around 2.4 μm²/s. While the specific effects of ions on the hydration force is complex and multifactorial, the different effects of ionic strength for bilayers on the two substrates observed suggest that ionic strength influences properties of the hydration layer which couples the bilayer to the substrate, rather than the lipid bilayer itself. That is, the properties of the hydration layer for supported lipid bilayers on the two substrates appear to vary and strongly influence the fluidic properties of the lipid bilayer.

### Extended-DLVO Model Analysis of Lipid-Substrate Interaction

The hydration layer between the supported lipid bilayer and the substrate can also be viewed as the cumulative effect of the forces stabilizing the system. From this viewpoint, the bilayer and substrate are treated as two parallel planes and the equilibrium separation distance (i.e., hydration layer thickness) corresponding to the minimum total interaction energy between the two planes is calculated by using an extended DLVO-type model that takes into account the van der Waals, double-layer electrostatic, and hydration forces. The hydration force is a short-range repulsive force between two hydrophilic surfaces and may be represented as an exponential decay function with a characteristic decay length, λ0, that describes the range of the hydration interaction energy.

The QCM-D measurement values from the SALB experiments show that the supported lipid bilayer on aluminum oxide has a relatively thicker hydration layer, which is due to confined interfacial water indicative of a strong hydration force.

In FIG. 5, the total interaction energy is presented as a function of separation distance for supported lipid bilayers on aluminum oxide or silicon oxide at varying decay lengths of the hydration force. In the calculations, the decay length is treated as the free parameter and it is assumed that the supported lipid bilayer has an equivalent hydration force on the two substrates. At 0.25 nm decay length, it is observed that the equilibrium separation distance for a supported lipid bilayer on aluminum oxide is predicted to be 1.97 nm and the corresponding total interaction energy decreases with increasing ionic strength from −36 ρJ/m² to −29 ρJ/m² (FIG. 5A). By contrast, on silicon oxide, a supported lipid bilayer is predicted to have an equilibrium separation distance of 2.41 nm at high ionic strength and the corresponding total interaction energy is around −7 ρJ/m² (FIG. 5B). Under low ionic strength conditions, the lipid-substrate interaction is predicted to be energetically unfavorable. From these results, one would predict that a supported lipid bilayer on silicon oxide has a greater separation distance than a bilayer on aluminum oxide. However, this prediction is not consistent with the experimental evidence (obtained herein), suggesting that the assumption that the decay lengths of the hydration force on the two substrates are equivalent is invalid. Rather, it appears that the decay length of the hydration force varies on the two substrates.

Based on these preliminary conclusions, the calculations were adjusted in order to be more consistent with experimental measurements of the bilayer thickness (i.e., 2 nm thicker on aluminum oxide than silicon oxide, which is attributed to confined interfacial water). In order to do so, decay lengths which provide experimentally reasonable estimates of the equilibrium separation distance were selected. In particular, the total interaction energy for a supported lipid bilayer on aluminum oxide was estimated for a decay length of 0.35 nm, yielding an equilibrium separation distance of 3.33 nm and a total interaction energy around −13 μJ/m² (FIG. 5C). The separation distance is compatible with the expected hydration layer thickness on aluminum oxide films based on the QCM-D measurement results. Likewise, for a supported lipid bilayer on silicon oxide, a decay length of 0.20 nm was used and a separation distance of 1.73 nm is predicted along with a total interaction energy around −13 ρJ/m² (FIG. 5D). Taken together, the extended-DLVO model calculations demonstrate that the range, of the hydration force for a supported lipid bilayer on aluminum oxide is appreciably greater than for a lipid bilayer on silicon oxide.

In summary, the extended-DLVO model calculations support that the hydration force for a supported lipid bilayer on aluminum oxide is comparatively stronger than for a bilayer on silicon oxide. The results highlight the utility of the SALB method to fabricate lipid bilayer coatings on solid supports with high surface hydration. Indeed, while a strong hydration force increases the challenge of bilayer fabrication, it also confers possible advantages by stabilizing lipid bilayers with thicker hydration layers due to the confined interfacial water.

Compared to supported lipid bilayers on silicon oxide, bilayers on aluminum oxide had appreciably thicker hydration layers. FRAP measurements further support that the hydration layer on aluminum oxide is tightly coupled to the substrate and in turn leads to hydrodynamic coupling with the bilayer, as reflected in a lower diffusion coefficient in this case than for bilayers on silicon oxide. The hydration force of a supported lipid bilayer on aluminum oxide has a much greater decay length, which means that the hydration force has a longer range likely due the confinement of water molecules at the interface. Consequently, the hydration force has an important steric factor that contributes to its repulsive nature by retarding the other interfacial forces, most notably the van der Waals force. As demonstrated herein, this issue can be overcome for lipids by using the SALB approach to deposit lipids in alcohol, followed by solvent-exchange to spontaneously form the supported lipid bilayer.

### Example 4

### Formation of a Hydration Layer/Lipid Bilayer on a Range of Substrates

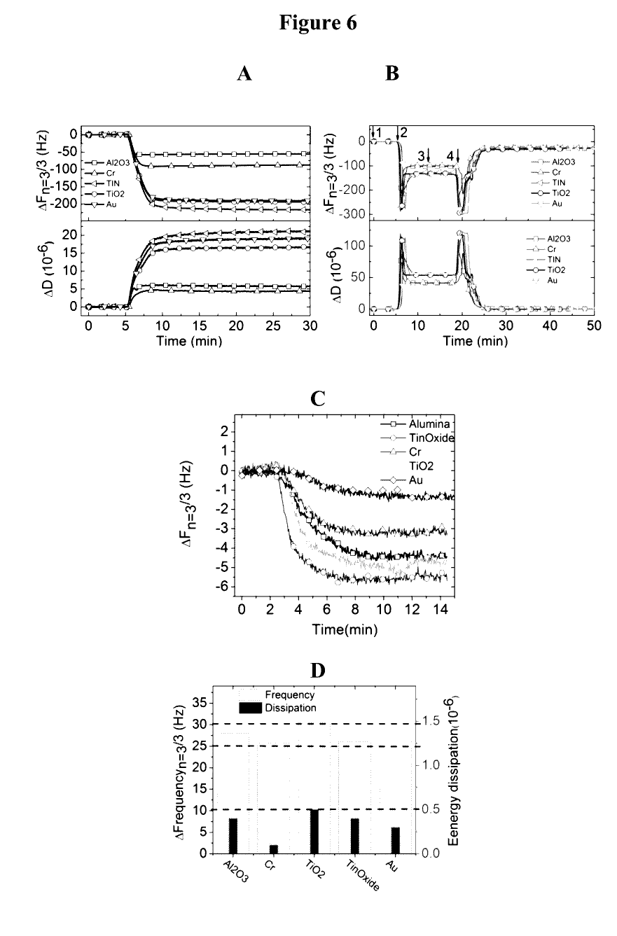

SALB experiments were conducted on a range of substrates including aluminum oxide (Al₂O₃), chromium (Cr), tin oxide (SnO), titanium oxide (TiO₂), and gold (Au). FIG. 6 compares the method on the different substrates. A lipid bilayer was formed on all the substrates.

### Example 5

### Establishment of a Lipid Range

It is generally accepted in the art that if a lipid bilayer forms with a method at a minimum lipid concentration that it will form for any concentration above the minimum and that the rate will be faster at higher lipid concentrations. Contrastingly, it was observed that there were strange formations that resulted at higher lipid concentration in various experiments mentioned herein. It was apparent that a bilayer only forms using this method within a range of lipid concentration. The range was established as follows.

### Quartz Crystal Microbalance-Dissipation.

A Q-Sense E4 (Q-Sense AB, Gothenburg, Sweden) instrument was employed to monitor changes in the resonance frequency (Δf) and energy dissipation (ΔD) of a 5 MHz, AT-cut piezoelectric quartz crystal, as previously described. The measurement data was collected at the n=3-11 overtones, and the reported values were recorded at the third overtone number (Δfn=3/3). All QCM-D experiments were performed under flow-through conditions at a flow rate of 50 μL·min⁻¹ using a peristaltic pump (Ismatec Reglo Digital). The temperature of the flow cell was fixed at 24.00±0.5° C.

### Epifluorescence Microscopy

Fluorescence imaging experiments were performed using an inverted epifluorescence Eclipse TE 2000 microscope (Nikon) equipped with a 60× oil immersion objective (NA 1.49), and an Andor iXon+EMCCD camera (Andor Technology, Belfast, Northern Ireland) camera. The acquired images consisted of 512×512 pixels with a pixel size of 0.267×0.267 μm. Rhodamine-modified phospholipid (0.5 wt %) was incorporated within the lipid mixtures in order to visualize the lipid assemblies. The samples were illuminated by a TRITC (rhodamine-DHPE) filter set with a mercury lamp (Intensilight C-HGFIE; Nikon Corporation).

Fluorescence Recovery after Photobleaching (FRAP) Measurements

Lipid mobility in the bilayer was measured by FRAP. After bilayer formation, a circular spot (d≈30 μm) was photobleached by a 532 nm, 100 mW laser beam. The bleaching time was ˜5 sec. The recovery was followed for 60 sec by time-lapse recording with 1 sec interval. FRAP data was analyzed using the Hankel transform method.

### Atomic Force Microscopy.

An NX-Bio atomic force microscope (Park Systems, Suwon, South Korea), combined with an Eclipse Ti optical microscope (Nikon, Tokyo, Japan), was employed to image SALB samples with contact mode imaging. A fresh silicon nitride, ultra sharp, AFM tip (μmesh, USA) was treated with oxygen plasma (Harrick Plasma), for 5 min, and sequentially rinsed with ethanol (70%), ultrapure water, and ethanol (70%) before drying with a gentle stream of nitrogen gas. Experiments were performed in an acoustic enclosure (Park Systems) with a temperature controller set at a constant temperature of 25° C. AFM imaging was performed on SALB samples immediately after QCM-D experiment and post-rinsing with 10 mM Tris buffer (150 mM NaCl, pH 7.5).

A typical SALB experiment was performed as follows (FIGS. 1B and 1D): first, buffer (10 mM Tris, 150 mM NaCl, pH 7.5) was injected into the QCM-D measurement chamber in order to establish the baseline for the frequency and dissipation signals. After 15 min stabilization, the organic solution (i.e., isopropanol) was injected (arrow 1), which leads to a dramatic shift in the baseline after a short transient change. During this stage, the solution does not contain lipid and the observed dramatic shift in the baseline is solely due to the density and viscosity difference between isopropanol and the buffer. After 15 min additional stabilization, a freshly prepared isopropanol solution containing the desired concentration of DOPC lipid was injected (arrow 2), which led to a decrease in the frequency due to adsorption of lipid at the surface. No changes in the dissipation were observed in this stage. At the final stage (arrow 3), the lipid solution (isopropanol+lipid) was replaced by aqueous buffer solution.

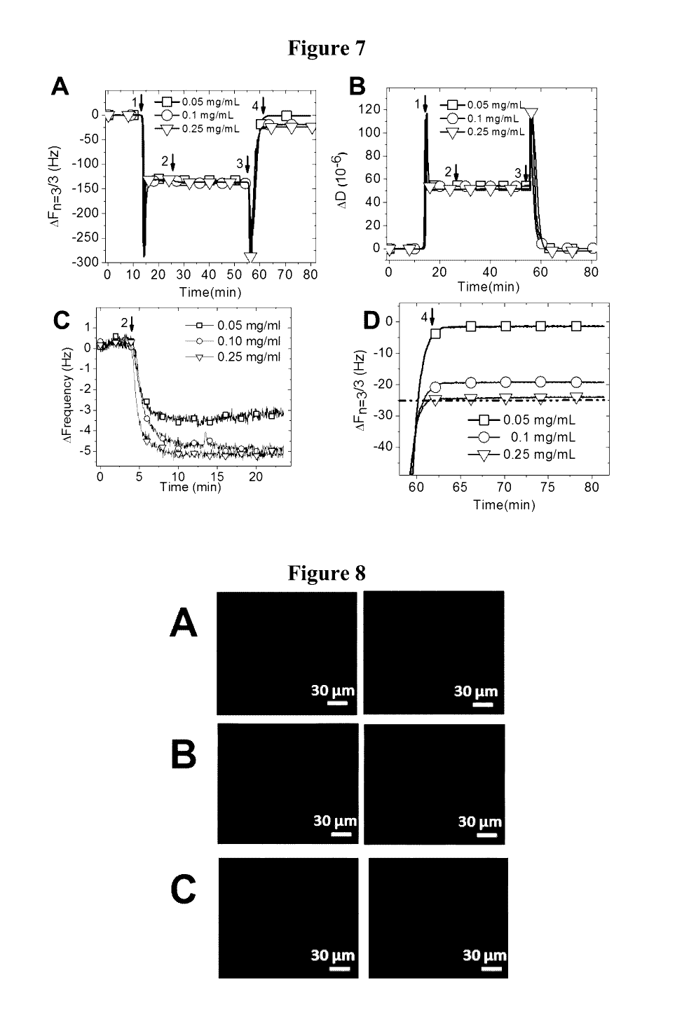

Using the SALB protocol, the minimal lipid concentration required to form a planar bilayer on silicon oxide was determined (FIG. 7). Three concentrations of DOPC lipid in isopropanol solution were selected: 0.05, 0.1 and 0.25 mg·mL-1. Independent of lipid concentration, there were general trends in the changes in frequency (FIG. 7A) and energy dissipation (FIG. 7B), suggesting that the changes occurred predominately due to the different solvent properties, including viscosity and density. In particular, a linear dependence was observed, a+bc (a and b are constants), of the lipid-induced frequency shift on lipid concentration, c, in solution (FIG. 7C). This shift is expected to be approximately proportional to the amount of adsorbed lipid. Furthermore, after the buffer replaced the isopropanol, the final changes in the QCM-D measurement signals varied depending on the lipid concentration (FIG. 7D). At 0.05 mg·mL-1 lipid, bilayer formation was not successful, as indicated by final changes in frequency and energy dissipation of −3 Hz and 0.07×10⁻⁶, respectively. This frequency shift is slightly lower than that observed after the lipid-isopropanol mixture injection (FIG. 7C), i.e., a part of the lipids seems to leave the surface when the isopropanol solution is replaced with aqueous buffer solution. Based on the Sauerbrey relationship between the change in frequency and adsorbed mass, the final mass of adsorbed lipids corresponds to 12% of the mass which is required to form a complete bilayer. At 0.1 mg·mL⁻¹ lipid, the final changes in frequency and energy dissipation were −19.2 Hz and 0.3×10⁻⁶, respectively, which corresponds to a bilayer which is between 75-80% complete. By contrast, at 0.25 mg·mL⁻¹ lipid, the final changes in frequency and energy dissipation were −25 Hz and 0.2×10⁻⁶, respectively. These values are consistent with complete bilayers formed at higher lipid concentrations as well as by the vesicle fusion method.

Epifluorescence microscopy images were also recorded in order to characterize the lipid assemblies in aqueous solution after formation by the SALB method, as shown in FIG. 8. For each lipid concentration, the left and right images are representative of the lipid assembly immediately after photobleaching and one min thereafter, respectively. At 0.05 mg·mL⁻¹ lipid concentration, isolated lipid structures were observed on the substrate, with relatively low surface coverage (FIG. 8A). At 0.1 mg·mL⁻¹ lipid concentration, there were microscopically homogenous bilayer patches although the bilayer was not microscopically homogenous across the entire field of view (100 μm×100 μm) (FIG. 8B). As with 0.05 mg·mL⁻¹ lipid concentration, there were also smaller lipid structures visible on the substrate. At 0.25 mg·mL-1 lipid concentration, a microscopically homogenous lipid structure was formed and fluorescence recovery after photobleaching (FRAP) analysis indicated a diffusion coefficient of 2.5 μm²·s⁻¹ (FIG. 8C), well within the expected range for a fluid, planar bilayer. Collectively, the epifluorescence microscopy results are consistent with the QCM-D measurements, and suggest that the SALB formation process involves a bilayer growth mechanism that starts with isolated lipid structures on the solid support.

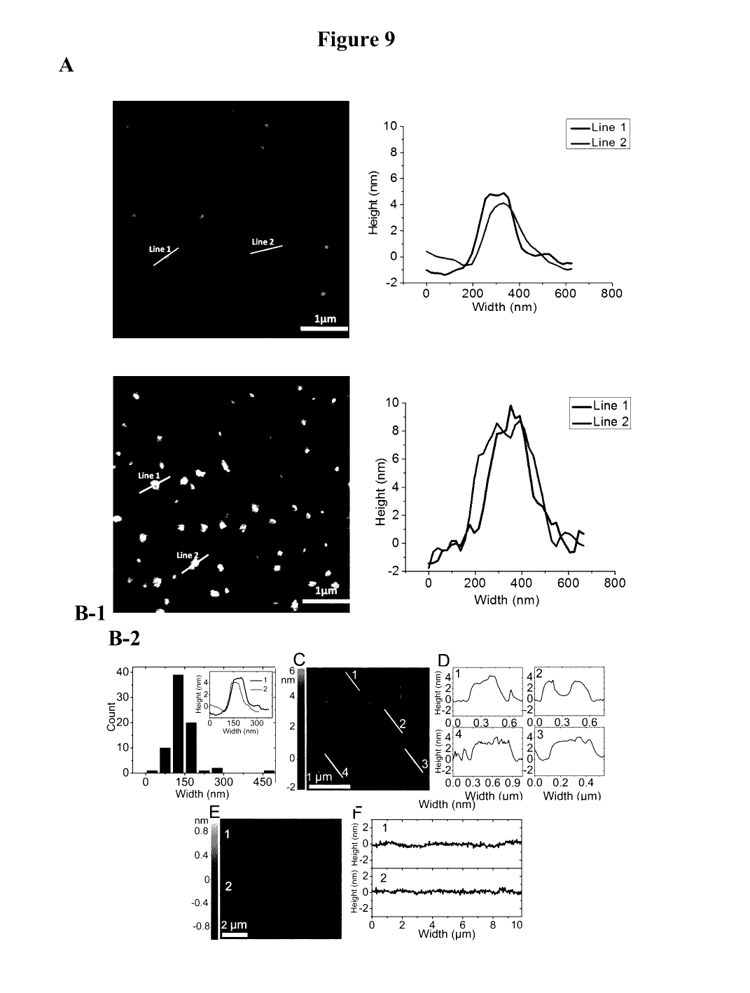

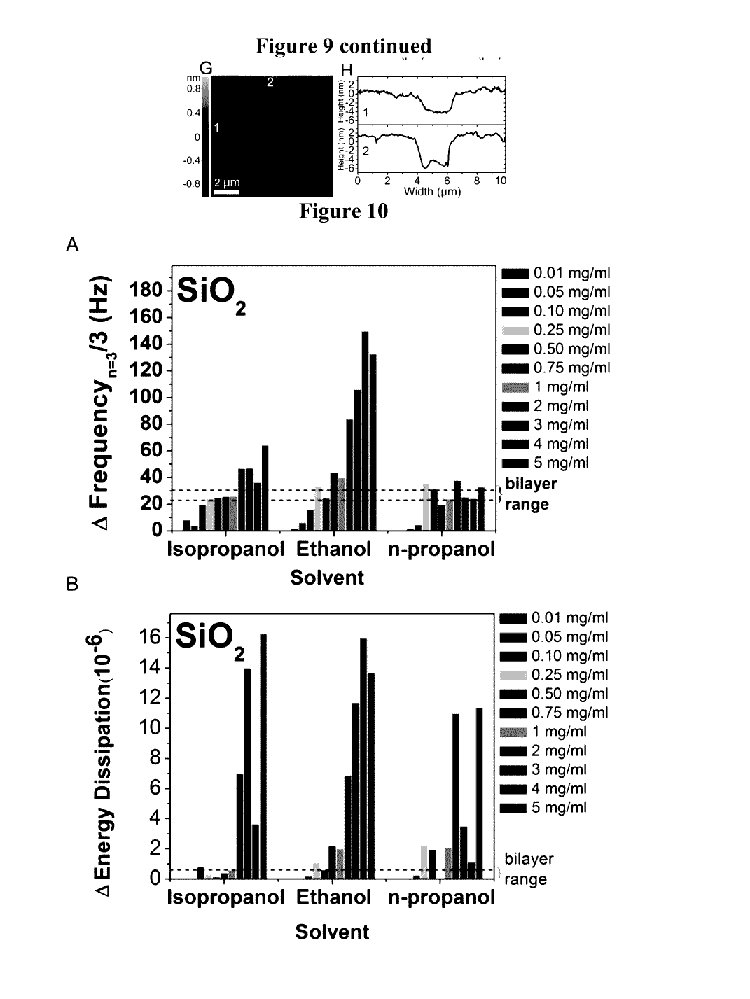

Atomic force microscopy (AFM) was used in order to determine the morphology of the isolated lipid structures observed in aqueous buffer solution at 0.05 and 0.1 mg·mL-1 lipid concentrations. A clue pointing to the mechanistic importance of these lipid structures is that they were also present when incomplete bilayers were formed. To prepare samples for AFM imaging, SALB experiments were performed on silicon oxide in the QCM-D measurement chamber. After completion of an SALB experiment at the desired starting lipid concentration, the SALB substrate was transferred to the AFM measurement chamber. This approach allowed us to establish a correlation between the AFM and QCM-D experimental results. At 0.05 mg·mL-1 lipid, there was a final change in frequency of −2 Hz, and AFM height profile analysis identified lipid structures with approximately 200 to 300 nm diameter and a height of around 4-5 nm (FIG. 9A). Previously, X-ray diffraction studies have measured that the DOPC bilayer thickness is approximately 3.6 nm. Considering that an ˜1 nm hydration layer stabilizes a planar bilayer on silicon oxide, the measured thickness is consistent with the structures being planar bilayer islands. At 0.1 mg·mL-1 lipid, there was a final change in frequency of −6 Hz. AFM height profile analysis indicated the presence of structures with approximately 200 to 300 nm diameter and 8 to 10 nm height (FIG. 9B). These structures may likely be double bilayer islands. The size-equivalence with respect to diameter between the single and double bilayers suggests there is a line tension which regulates the size of the small lipid islands in the absence of sufficient lipid to propagate bilayer growth and completion. For 0.1 mg/mL lipid, mesoscopic lipid bilayer fragments of nonuniform shape were observed with similar height characteristics (FIG. 9C-D). The size ranges of the isolated lipid structures formed at 0.05 and 0.1 mg/mL lipid concentrations, respectively, support that the difference in absolute mean fluorescence intensity of diffraction-limited spots in each case is primarily due to variation in the surface area of planar bilayer fragments. For 0.25 mg/mL lipid, a complete planar bilayer was formed based on the appearance of a smooth homogenous surface (FIG. 9E-F). To confirm the formation of a planar bilayer, a 2×2 μm square segment of the lipid layer was removed by AFM tip scanning and the corresponding height changes indicated that the lipid structure has a thickness of around 4-5 nm (FIG. 9G-H). According to previous X-ray diffraction studies, the DOPC lipid bilayer thickness is approximately 3.6 nm. Considering that a ˜1 nm-thick hydration layer stabilizes a planar bilayer on silicon oxide, the thickness measured is consistent with planar bilayer structures.

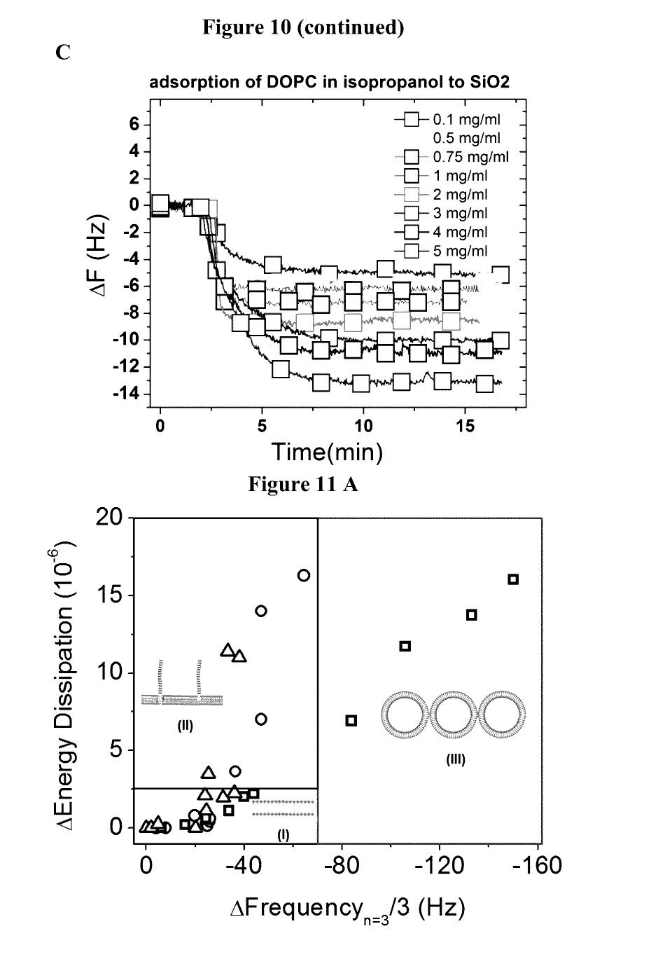

The effect of lipid concentration on the final outcomes of the SALB experiments was also investigated over a wider lipid concentration range up to 5 mg/ml lipid and in different organic solvents (isopropanol, ethanol and n-propanol), as presented in FIG. 10. The corresponding adsorption kinetics of lipids in isopropanol are shown in the supporting information (FIG. 10C; for the other solvents, the results are similar). The QCM-D-measured characteristics of the lipid structures obtained in the end of the SALB procedure are presented in FIG. 10. The reported values correspond to the final measurement after completion of the SALB procedure, i.e., to the lipids on silicon oxide in aqueous buffer solution. Bilayer formation was defined based on final changes in frequency and energy dissipation between −25 and −30 Hz and less than 0.5×10⁻⁶, respectively. The optimal lipid concentration range to form planar bilayers was determined to be between 0.1 and 0.5 mg·mL⁻¹ largely independent of the type of organic solvent. Interestingly, at higher lipid concentrations (2 mg·mL⁻¹ or greater), there were significant deviations in the final measurement responses, as compared to typically expected values for planar bilayers.

During the SALB procedure, within the tested lipid concentration range (between 0.1 and 5 mg·mL⁻¹), the lipid adsorption process rapidly reached steady state, and there was a linear dependence, a+bc (FIG. 7C), between the amount of adsorbed lipid in organic solution and the lipid concentration in bulk solution. The observation of steady state indicates either saturation of the overlayer or equilibrium between the solution and overlayer. Equilibrium would be the expected reason but saturation was also investigated. In both cases, the finding that the uptake is linear and is appreciable can hardly be explained by assuming that the size of inverted micelles is independent of lipid concentration and using conventional models of adsorption kinetics. In other words, this finding indicates the inverted micelle size depends on lipid concentration).

In isopropanol and n-propanol, high lipid concentrations led to the formation of lipid structures that had changes in frequency within the range of a planar bilayer (−25±10 Hz), albeit with large changes in energy dissipation up to 12×10⁻⁶ (FIG. 10, isopropanol and n-propanol). A small change in frequency and corresponding large change in energy dissipation has not been generally observed in surface-adsorbed lipid assemblies in aqueous buffer solution (e.g., intact vesicles, planar bilayer). A more appropriate example is filamentous virus phage particles, which exhibit similar behavior, as observed herein at high lipid concentration in isopropanol and n-propanol. In the present case, it is possible that the filamentous structures correspond to wormlike micelles which can form upon addition of water to lecithins in organic solvent. In the SALB experiments, this process is analogous to step 3, e.g., FIG. 7A. Interestingly, in the SALB experiments, FRAP analysis identified that there is also a fluid bilayer on the substrate which underlies the filamentous structures, e.g., 5 mg·mL⁻¹ lipid in isopropanol. By contrast, in ethanol, high lipid concentrations formed phospholipid assemblies with viscoelastic properties that are more representative, of intact vesicle layers than planar bilayers, based on large changes in frequency and dissipation greater than −80 Hz and 4×10⁻⁶ (FIG. 10, ethanol). Compared to the other SALB experiments, FRAP analysis indicated that the corresponding phospholipid assemblies have low mobility in the ethanol case and a high immobile fraction, both of which are consistent with intact vesicles.

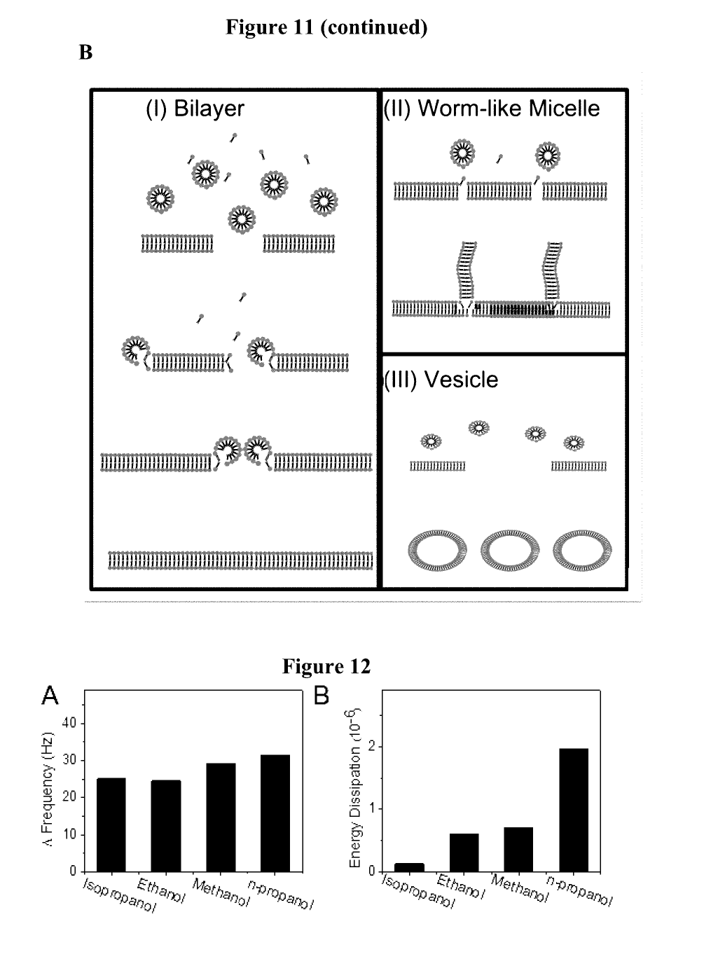

In FIG. 11, the final QCM-D measurement values for the SALB experiments are reported independently of lipid concentration. Based on the final changes in frequency and energy dissipation of the lipid structures in aqueous buffer solution, the results of the SALB formation process were classified into different categories. Following addition of aqueous buffer solution, the adsorbed lipids may self-assemble to form a planar bilayer if there is sufficient excess lipid in the bulk solution (FIG. 11, I). If there is insufficient lipid in the bulk solution, then the process may terminate before completion. Depending on the experimental conditions (e.g., lipid concentration and type of organic solvent), the SALB formation process may cause the formation of lipid structures with properties characteristic of filamentous structures (FIG. 11, II) or intact vesicles (FIG. 11, III). Interestingly, in the intact vesicle region, there is linearity of the relative changes in frequency and dissipation. Taken together, the experimental findings support that the SALB method is a bottom-up approach such that the preorganization of adsorbed lipids in organic solvent before aqueous buffer solution induces bilayer formation via surface nucleation at bilayer islands.

Based on these empirical observations, a basic mechanism of the SALB formation process (FIG. 11B) was proposed without being limited to any particular theory. Within the tested lipid concentration range (between 0.1 and 5 mg/mL), there was a linear dependence between the amount of adsorbed lipid in isopropanol and the lipid concentration in bulk solution. After addition of aqueous buffer solution, the attached inverted micelles appear to decompose and form bilayer islands. As observed by the AFM experiments in FIG. 8, adsorbed lipids can form bilayer islands, even at low lipid concentration. Based on the final QCM-D measurement responses in aqueous buffer solution after the SALB experiments were completed, the mass of adsorbed lipids arising from the decomposed micelles likely corresponds to at least ˜10-20% of the lipid necessary to form a planar bilayer. Therefore, lipid adsorption in organic solvent is an important step to influence the density of bilayer islands on the surface and accordingly promotes bilayer formation. Indeed, the bilayer islands serve as nucleation sites to propagate bilayer growth due to adsorption of additional lipids from the bulk solution. Following this line, it would be expected that bilayer formation is improved with increasing lipid concentration in solution. However, the SALB experiments indicate there is an optimal concentration range for bilayer formation.

When aqueous buffer solution is introduced into the measurement chamber to replace the organic solution, there is a transient mixing period as the water fraction increasingly grows. During this period, monomers and inverted micelles likely undergo phase transitions and self-assemble into monomers and micelles. In order to complete the SALB formation process, the lipid monomers/micelles in solution may adsorb onto the substrate and fuse with the bilayer islands (FIG. 11B, Case I). Depending on the density of bilayer islands, the findings support the idea that the nucleation process may occur in two- or three-dimensions. At a low density of islands, lipid fusion with the bilayer islands can form a complete planar bilayer. By contrast, at a high density of islands, the QCM-D measurement values suggest that filamentous structures can also form starting from adsorbed lipids originally in n-propanol and isopropanol (FIG. 11B, Case 2). This behavior is likely due to a combination of solvent properties and the lipid concentration. Indeed, in contrast to the SALB experiments performed using n-propanol and isopropanol, experiments performed using ethanol results in lipid layers with properties characteristic of an intact vesicle adlayer (FIG. 11B, Case 3). Hence, the nucleation process, and its dependence on different experimental parameters including lipid concentration and type of organic solvent, can give rise to multiple lipid assemblies, including complete planar bilayers within an optimal concentration range. The optimal range is likely due to an interplay of island density (sufficiently low to exclusively promote two-dimensional nucleation) and lipid concentration in bulk solution (provide enough lipid to fuse with the bilayer islands and form a planar bilayer). Overall, the experimental findings provide a framework to understand the mechanism of the SALB formation process, optimize the SALB procedure and interpret measurement data collected when using the SALB method.

### Example 6

### Effects of Alcohol and pH on the Method

The effect of organic solvent type on bilayer formation kinetics on silicon oxide was examined. In addition to isopropanol, three other organic solvents, which are 100% miscible with water, were selected, including ethanol, n-propanol, and methanol. In all cases, the SALB formation process was able to form planar bilayers using an identical procedure as described above. However, the quality of the bilayers formed is different depending on the solvent used (FIGS. 12A,B). The final Δf values for isopropanol, ethanol, methanol and n-propanol were 25.4, 25.3, 29.3 and 32.6 Hz, respectively, and the corresponding AD values were 0.2×10-6, 0.6×10-6, 0.7×10-6 and 1.96×10-6, respectively. Hence, the findings support that isopropanol is the best solvent to form a planar bilayer.

The effect of pH on bilayer formation kinetics on silicon oxide was examined. 0.5 mg/mL DOPC lipid was used and the buffer solution contained 10 mM Tris and 150 mM NaCl with varying pH. Bilayer formation was defined by frequency shifts between −23 and −28 Hz and energy dissipation shifts less than 1×10⁻⁶. The results are listed in Table 1. Optimal bilayer formation was achieved in the pH range of 3-9, demonstrating that the method is able to work over a wide range of pH conditions. Together with the effects of pH on the bilayer-substrate interaction energy, this approach enables precise contra over bilayer properties which cannot be achieved with alternative fabrication techniques.

TABLE 1
Effect of Solution pH on Formation of Supported
Lipid Bilayers on Silicon Oxide by the method.

| | ΔFrequency | ΔEnergy Dissipation |
|---|---|---|
| pH | (Hz) | (10⁻⁶) |
| 2 | −30.8 | 2.15 |
| 3 | −26.7 | 0.1 |
| 4 | −24.9 | 0 |
| 5 | −25.8 | 0.1 |
| 6 | −23.7 | 0 |
| 7 | −27.9 | 0 |
| 8 | −24.6 | 0.9 |
| 9 | −27.4 | 0.8 |
| 10 | −39.0 | 1.9 |
| 11 | −18.4 | 3 |
| 12 | −11.0 | 3.2 |

### Example 7

### Lateral Lipid Diffusion of SALB Bilayers

Fluorescence Microscopy and Fluorescence Recovery after Photobleaching (FRAP).

Fluorescence microscopy imaging of silica-supported planar bilayers with 0.5 wt. % rhodamine-modified phospholipid was performed by using an inverted epifluorescence Eclipse TE 2000 microscope (Nikon) equipped with a 60× oil immersion objective (NA 1.49), and an Andor iXon+EMCCD camera (Andor Technology, Belfast, Northern Ireland). The acquired images consisted of 512×512 pixels with a pixel size of 0.267×0.267 μm. The samples were illuminated through a TRITC (rhodamine—DHPE) filter set by a mercury lamp (Intensilight C-HGFIE; Nikon Corporation). For FRAP measurements, a 30 μm-wide circular spot was photobleached with a 532 nm, 100 mW laser beam, followed by time-lapse recording. Diffusion coefficients were determined by the Hankel transform method (Jonsson et al. Biophysl J, 2008, 95 (11), 5334-5348), along with immobile fraction.

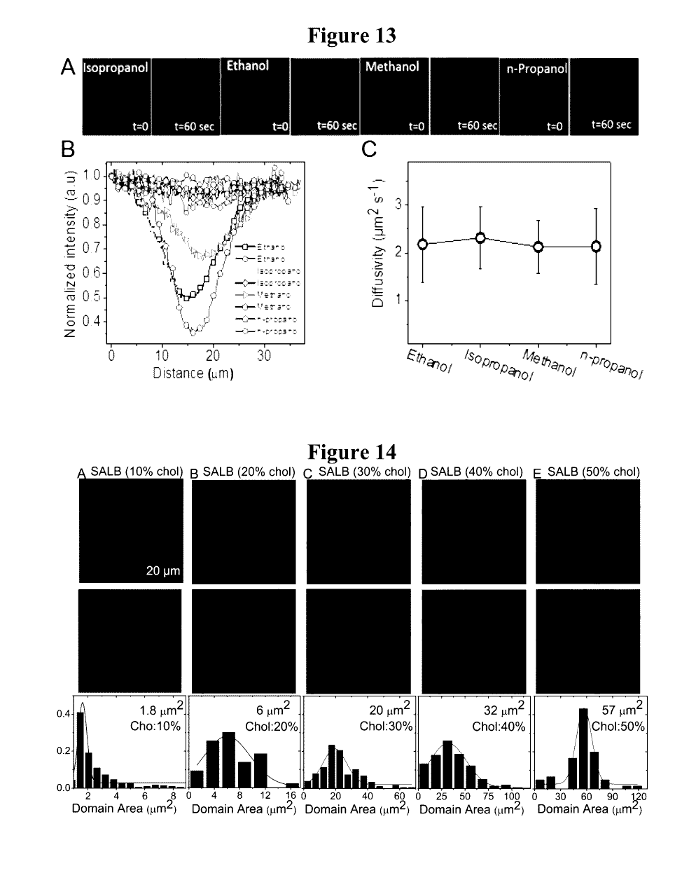

In order to further analyze the physical properties of planar bilayers formed via the SALB method with different organic solvents, homogeneity and fluidity of bilayers were characterized by fluorescence microscopy and fluorescence recovery after photobleaching (FRAP) (FIG. 13). FIG. 13A shows bilayer images immediately after photobleaching (left) and after one minute recovery (right). The intensity profiles show that the fluorescent recovery was almost complete in one minute, indicating that the lipids are laterally mobile. (n=5) (FIG. 13B). The FRAP data was analyzed using the Hankel transform method, yielding an average diffusion coefficient of 2 μm²/s in all cases (FIG. 13C). In addition, the mobility values are in good agreement with the range of diffusivity expected for a fluid supported bilayer. The bilayers generally have 96% or greater mobile fractions. The bilayer formed via n-propanol had a 90% mobile fraction, which is consistent with the appreciably larger change in energy dissipation as well as the appearance of lipid aggregates on top of the bilayer in the fluorescence microscopy images. Overall, it is determined that the type of organic solvent alcohol is an important parameter to optimize SALB formation, and preferably isopropanol is suitable for SALB formation.

### Example 8

### Formation of Cholesterol-Rich Supported Membranes

### Sample Preparation

A zwitterionic lipid, 1,2-dioleoyl-sn-glycero-3-phosphocholine (DOPC), cholesterol (ChoI), and fluorescently labeled lipid, 1,2-dioleoyl-sn-glycero-3-phosphoethanolamine-N-(lissamine rhodamine B sulfonyl) (ammonium salt) (Rhodamine-DHPE), were purchased from Avanti Polar Lipids (Alabaster, Ala.). Immediately prior to the experiment, the lipid powder was dissolved in isopropanol at 10 mg·mL⁻¹ lipid concentration, mixed to the desired DOPC:ChoI molar ratio, and then diluted to 0.5 mg·mL⁻¹ lipid concentration. The aqueous buffer solution was 10 mM Tris buffer solution [pH 7.5] with 150 mM NaCl. In fluorescence microscopy experiments, the lipid composition also contained 0.5 wt % Rhodamine-DHPE. Small unilamellar vesicles were prepared as follows: dried lipid films were rehydrated in aqueous buffer solution at a nominal lipid concentration of 5 mg·mL⁻¹. The hydrated lipid films were then extruded through 50-nm diameter track-etched polycarbonate membranes in order to form small unilamellar vesicles, as previously described 33.

### Epifluorescence Microscopy

Epifluorescence microscopy imaging was performed using an inverted epifluorescence Eclipse TE 2000 microscope (Nikon) equipped with a 60× oil immersion objective (NA 1:49), and an Andor iXon+EMCCD camera (Andor Technology, Belfast, Northern Ireland). The acquired images had dimensions of 512×512 pixels with a pixel size of 0.267×0.267 μm. The samples were illuminated through a TRITC (Rhodamine—DHPE) filter set by a mercury lamp (Intensilight C-HGFIE; Nikon Corporation). For fluorescence recovery after photobleaching (FRAP) analysis, a 30 μm-wide circular spot was photobleached with a 532 nm, 100 mW laser beam, followed by time-lapsed recording. The bleaching time was 5 sec. The recovery was followed for 60 sec at 1 sec intervals, and diffusion coefficients were computed using the Hankel transform method (Jonsson et al. Biophys J 2008, 95, (11), 5334-5348). All image processing was done using ImageJ software. For bilayer formation using the SALB procedure, commercially available microfluidic flow cells (stick-Slide I0.1 Luer, Ibidi, Munich, Germany) were employed, with an injection flow rate of 50 μL·min⁻¹.

### Atomic Force Microscopy

An NX-Bio atomic force microscope (Park Systems, Suwon, South Korea), combined with an Eclipse Ti optical microscope (Nikon, Tokyo, Japan), was employed to image SALB experimental samples in contact mode. An ultra-sharp, silicon nitride BioLever mini cantilever tip (Olympus, Tokyo, Japan) was used for all experiments. The tip has a tetrahedral shape, 110 kHz resonance frequency, and 0.09 N·m⁻¹ spring constant. Prior to the experiments, the tip was subjected to oxygen plasma treatment at maximum radio-frequency power (Harrick Plasma, Ithaca, N.Y.) for 5 min, and sequentially rinsed with ethanol (70%), ultrapure water, and ethanol (70%) before finally drying with a gentle stream of nitrogen air. Experiments were conducted in an acoustic enclosure (Park Systems) with a temperature controller set at a constant temperature of 25° C. AFM imaging was done on SALB samples immediately after QCM-D experiment and post-rinsing with 10 mM Tris buffer [pH 7.5] with 150 mM NaCl.

### Quartz Crystal Microbalance-Dissipation

A Q-Sense E4 instrument (Q-Sense AB, Gothenburg, Sweden) was employed to monitor the adsorption kinetics of lipids onto silicon oxide- and gold-coated 5 MHz, AT-cut piezoelectric quartz crystals. Changes in frequency (ΔF) and energy dissipation (ΔD) were recorded as functions of time, as previously described (Keller et al. Biophys J 1998, 75, (3), 1397-1402). The measurement data was collected at the n=3-11 overtones, with the reported values having been recorded at the third overtone. The normalized values (Δfn=3/3) are reported. All samples were introduced at a flow rate of 50 μL·min⁻¹ using a peristaltic pump (Ismatec Reglo Digital) under continuous flow conditions. The temperature of the flow cell was fixed at 24.00±0.5° C. Before the experiments, the sensor surfaces were treated with oxygen plasma at maximum radio-frequency power (Harrick Plasma) for 1 min immediately before use.

Using the SALB method, DOPC/ChoI lipid bilayers were prepared containing variable fractions of cholesterol (between 0 and 50 mol % ChoI) on a silicon oxide substrate. A precursor mixture of phospholipid and ChoI in isopropanol solution was incubated in the measurement chamber for a minimum of 10 min, and then aqueous buffer solution was flowed-through the chamber to facilitate solvent-exchange. A small fraction (0.5 wt %) of Rhodamine-DHPE fluorescent dye was included in the precursor mixture in order to visualize the bilayer phase formed on the substrate by epifluorescence microscopy. Preliminary quartz crystal microbalance with dissipation monitoring (QCM-D) measurements reveal that the final frequency and energy dissipation shifts are −25.3±3.4 Hz and 0.7±0.7×10⁻⁶, respectively, which, based on previous reports, confirms that the SALB process produces single supported bilayers at the substrate surface comparable in surface density to those formed by vesicle fusion and by the Langmuir-Blodgett method.

Typical epifluorescence micrographs (100×100 μm) are presented in FIG. 14 for the supported membranes formed on silicon oxide following completion of the SALB procedure. The micrographs reveal the formation of a membrane phase, characterized by a uniform fluorescence intensity consistent with the formation of a single lipid bilayer. In addition to the phospholipid-rich phase, there are dye-excluded circular spots distributed randomly throughout the membrane phase (FIG. 14), which are largely monodisperse in single samples and stable over long periods of time Because Rhodamine-DHPE partitions preferentially in the fluid phase, the dye-decorated surroundings was tentatively ascribed to the cholesterol-depleted, phospholipid rich fluid phase and the dark spots to the cholesterol-enriched dense phase. In order to determine if the phase-separating supported, membrane morphologies exhibit long-range lateral diffusion (and fluid character), a microscope-based FRAP experiment was carried out. In FIG. 14, upper and lower micrographs are captured at t=0 and 1 min after bleaching, respectively. For all cholesterol concentrations, near-complete recovery of the photobleached spot suggests that the dye-laden, cholesterol-depleted phase is indeed laterally fluid.

This assignment is further consistent with the fact that the sizes of the dye-depleted domains increase with the cholesterol content: With increasing ChoI fraction in the precursor mixture, the average domain size increases from 1.8 μm² for 10 mol % ChoI to 57 μm² for 50; mol % ChoI. As expected, no dye-excluded domains are apparent in a single component DOPC lipid bilayer also formed by the SALB procedure (data not shown). DOPC/ChoI membranes were prepared on the same substrates through conventional vesicle fusion. Although the preparation of uniform bilayer samples with high cholesterol concentrations (>20%) was erratic and irreproducible, supported membrane samples obtained using vesicles containing 20 mol % ChoI or less also revealed dark circular domains similar to those observed in samples prepared by the SALB process.

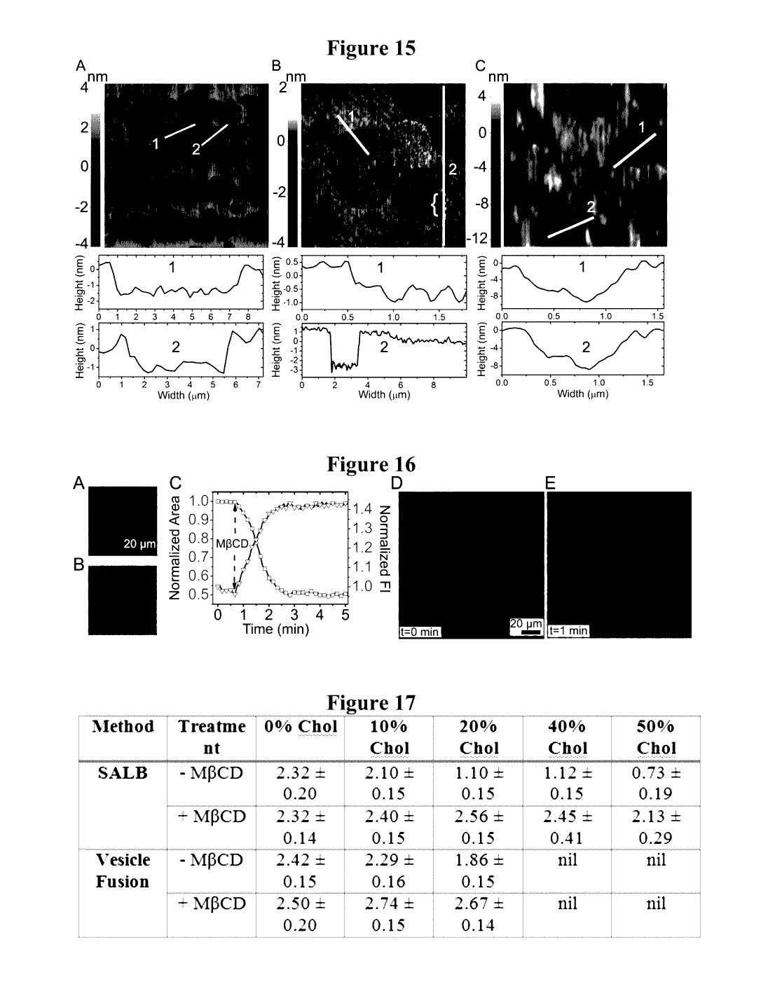

In order to further characterize the dye-excluded domains, independent atomic force microscopy (AFM) experiments were conducted. FIG. 15 shows representative AFM micrographs for SALB-prepared DOPC/ChoI bilayers containing 30 mol % ChoI fraction. Consistent with large-area fluorescence images, the dye-excluded domains are nearly circular in shape, occasionally present as two or more coalescing domains. A line scans across the domain edges show that the phospholipid-rich phase is ˜1.5 nm thicker than the dye-excluded domains. The thickness of the phospholipid-rich phase was determined to be ˜4.5 nm so the dye-excluded domains are ˜3 nm thick consistent with the presence of cholesterol enriched membrane domain. Additional support for the presence of cholesterol-rich phase in circular domains is obtained through a cholesterol extraction assay. Using 1 mM methyl-β-cyclodextrin (MβCD)—which is a cyclic oligosaccharide that removes ChoI from lipid bilayers a height difference between the surrounding membrane phase and the dye-excluded domains was found to substantially increase to between 5 and 8 nm (FIG. 15C). This finding indicates that MβCD treatment removes ChoI from the dye-excluded domains and also appears to perturb the edge of the membrane phase surrounding the ChoI-rich domains. Taken together, the AFM data lend strong, direct support to the foregoing inference that the dye-excluded domains are highly enriched in cholesterol and not voids.

Interestingly, fluorescence micrographs of a 50 mol % ChoI-containing lipid bilayer before and after 1 mM MβCD treatment further demonstrate significant alterations in membrane properties in the phospholipid-rich phase as well (FIGS. 16A-B). Time-resolved measurements of the fluorescent properties of the lipid bilayer show that the surface area of the phospholipid-rich phase decreases by ˜50% upon treatment, and there is a corresponding increase in the normalized fluorescence intensity per unit of surface area by ˜46% (FIG. 16C). Hence, at elevated cholesterol content (50 mol %), MβCD treatment removes cholesterol from both the dye-excluded domains and the phospholipid-rich phase. This suggests that at high cholesterol contents in the so-called β phase, cholesterol associates with the lipids in a manner that produces a unique phase co-existence between cholesterol-lipid complexes in one phase and free cholesterol associated with the phospholipid-rich phase in the other. Note also that the original shapes of the dye-excluded domains is retained after cholesterol removal, presumably because reduced bilayer density due to cholesterol extraction) and limits on lateral expansion preclude the necessary spreading of the remainder of the supported bilayer. In order to measure the lateral fluidity of the phospholipid-rich phase before and after cholesterol removal, fluorescence recovery was carried out after photobleaching (FRAP) experiments and the results were analyzed by the Hankel transform method. Representative fluorescence micrographs, presented for 50 mol % ChoI-containing lipid bilayer after MβCD treatment (FIGS. 16D-E), confirm the retention of long-range fluidity and lateral fluidity of the residual, cholesterol-depleted membrane.

The FRAP experimental results for all membrane compositions before MβCD treatment are listed in (FIG. 17). At low ChoI fractions (0-10 mol %) in the precursor mixture, the lateral diffusion coefficient for lipid bilayers was around 2.2 μm²·s⁻¹. With increasing ChoI fraction (20-40 mol %), the diffusion coefficients are approximately 1.1 μm²·s⁻¹. Finally, at high ChoI fraction (50 mol %), the diffusion coefficient is lowered to 0.8 μm·s⁻¹. Following MβCD treatment, the diffusion coefficient of all lipid bilayers is between 2.2 and 2.6 μm²·s¹ (FIG. 17). The results support that MβCD treatment removes ChoI from the phospholipid-rich phase, and enabled quantitative determination of the mole fraction of ChoI in the lipid bilayer as explained below.

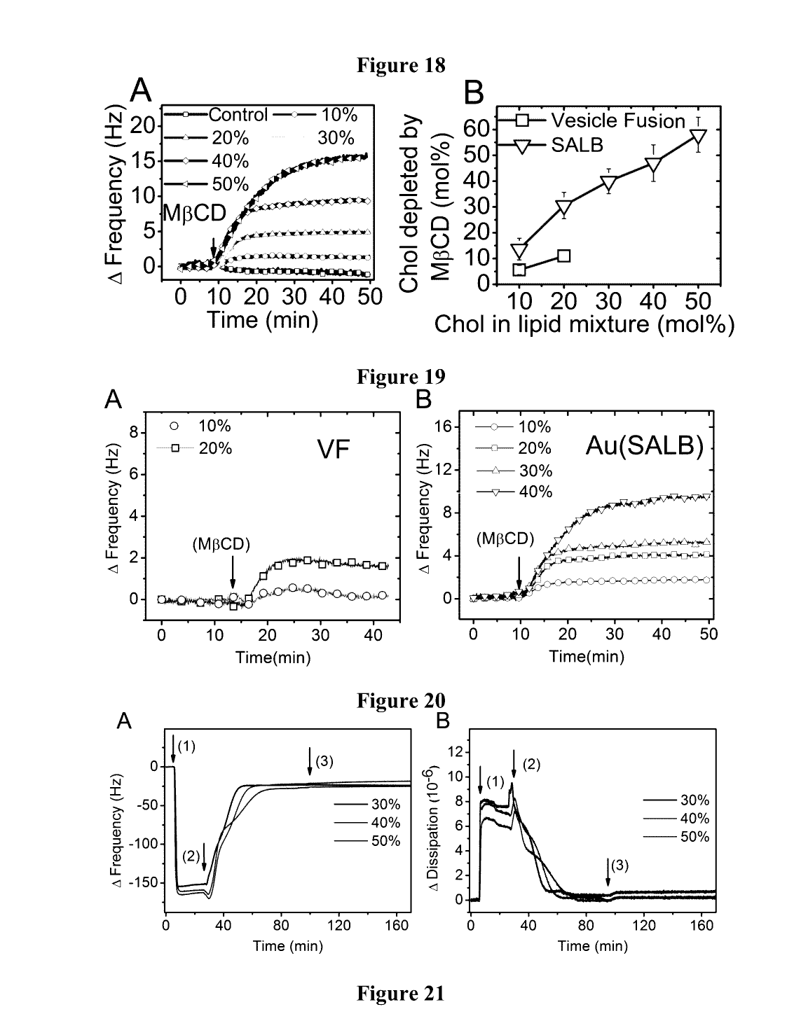

QCM-D tracking allows measurements of the negative frequency shift (ΔfBilayer) associated with a planar lipid bilayer on silicon oxide. The bilayers have low energy dissipation (ΔDBilayer<1×10⁻⁶) so the frequency shift is converted into adsorbed mass (ΔmBilayer) based on the Sauerbrey relationship. Assumption that the bilayer mass represents the sum of DOPC lipid (ΔmLipid) and ChoI (ΔmChoI) masses is made. After bilayer formation, 1 mM MβCD was then added in order to observe the positive frequency shift (ΔfChoI ∝ΔmChoI) associated with ChoI removal. The results of the MβCD treatment step are presented in FIG. 18A. The initial baseline values are the normalized frequency shifts for the bilayers (defined as ΔfBilayer=0). With increasing ChoI fraction in the precursor mixture, ΔfChoI increased proportionally and demonstrated that the cholesterol fraction in the bilayer can be tuned according to the cholesterol fraction in the precursor mixture. The kinetics of ChoI removal show first-order features that are consistent with previous reports. In order to calculate the mole fraction of cholesterol in the bilayer, the ΔfLipid based on the difference between Δf Bilayer and ΔfChoI, is determined and applied using the Sauerbrey relationship, taking the molecular weights of DOPC lipid and cholesterol into account.

Overall, the calculated mole fractions of ChoI in the bilayer demonstrate that the SALB procedure forms ChoI-enriched bilayers, which reflect the starting precursor compositions (FIG. 18B). There is a nearly linear correspondence between the ChoI fraction in the precursor mixture and the ChoI fraction in the bilayer, with a slight increase in the ChoI fraction in the bilayer. Importantly, the SALB procedure was also successful at forming supported bilayers on gold with similarly high levels of ChoI (FIG. 19). For comparison, planar bilayers formed from vesicles containing up to 20 mol % ChoI (i.e., the highest ChoI fraction at which vesicles can still rupture spontaneously) and measured the ChoI fraction in these bilayers by the same approach. Strikingly, the ChoI fraction in these bilayers was appreciably lower than in the precursor vesicles and a maximum fraction of ChoI in the bilayers was approximately 10 mol % (see FIG. 18B and FIG. 19).

In order to form planar bilayers with greater ChoI fractions via vesicle rupture, an amphipathic, α-helical (AH) peptide was used in order to rupture a layer of adsorbed vesicles containing between 30-50 mol % ChoI (FIG. 20). It was determined that the ChoI in the bilayer formed is intractable to MβCD treatment (no ChoI removal), suggesting that either there is no ChoI in the bilayer formed via this route or more likely that the peptide interferes with membrane properties. Taken together, the data support that the SALB procedure can form planar lipid bilayers containing high fractions of ChoI (at least five-fold greater than alternative vesicle fusion methods), and that ChoI is present in both the phospholipid-rich phase and cholesterol-rich domains at elevated concentrations.

By inducing supported bilayer formation via solvent-exchange, intrinsic challenges imposed by vesicle configurations (e.g., compositional heterogeneity, vesicle stability, and high bending rigidities) are bypassed, allowing the formation of supported bilayers through solvent-assisted lipid self-assembly. To some extent, the strategy also shares a common feature with Langmuir-type transfer processes, i.e., lamellar phase lipid organization at an interface due to water contact, albeit there is one key advantage of the SALB approach for studying phospholipid-cholesterol mixtures. The formation of lamellar phase lipid structures directly precedes to the bilayer configuration, and the corresponding self-assembly process is also influenced by molecular interactions between the two leaflets. Several new findings related to phospholipid-cholesterol mixtures in supported bilayers were found.

In fluid-phase DOPC containing lipid bilayers, cholesterol is located within the fluidic phospholipid-rich phase as well as in micron-scale cholesterol bilayer domains. The effects of cholesterol on the phospholipid-rich phase were consistent with expectations, e.g., decreased fluidity with increased cholesterol fraction. The ability of the SALB approach to prepare supported membranes containing high cholesterol concentrations up to the solubility limit of cholesterol in the lipid phase should prove useful in characterizing the high-concentration β-phase. The approach should also enable characterization of cholesterol bilayer domains (CBDs), which appear to be produced beyond the solubility limit when cholesterol crystallizes within the two-dimensional lipid environment. These CBDs are believed to be precursors of crystalline cholesterol, yet remain dynamic which might explain their susceptibility to MβCD treatment described here. Furthermore, crystalline deposits on silicon oxide are attempted by performing the SALB procedure from cholesterol alone and without DOPC lipid. In this case, no adsorbed layer was formed after solvent-exchange, which is again consistent with the fact that CBDs only form in conjunction with phospholipid-rich phases.

Regarding, the molar fraction of cholesterol in the supported bilayers, direct measurement of this value by QCM-D was made. Specifically, the mass of the supported bilayer containing DOPC lipid and cholesterol is determined, removed the cholesterol via MβCD treatment, and then the mass of the remaining adsorbed lipid determined again. To validate this approach, epifluorescence microcopy and AFM measurements verified that MβCD treatment removes cholesterol from both the phospholipid-rich phase and the cholesterol bilayer domains, and the desorption kinetics show first-order features. Radhakrishnan et al. previously showed that the mono-exponential decay is related to the chemical activity of cholesterol and the stoichiometric composition of condensed complexes of cholesterol and phospholipid (Biochem 2000, 39, (28), 8119-8124). After MβCD treatment, the residual lipid bilayers had a nearly consistent rate of lateral lipid diffusion, which agrees well with recent work. Furthermore, MβCD treatment had no effect on DOPC lipid bilayers, thus confirming its specific removal of cholesterol.

Importantly, this approach allows determination that the fraction of cholesterol which can be incorporated into the supported bilayer is more than 5 times greater than that permissible with conventional techniques for supported bilayer formation (e.g., vesicle fusion). The molar fraction of cholesterol in supported bilayers formed by the vesicle fusion method was appreciably lower than that of the precursor vesicles, although the molar fraction of the supported bilayer and precursor vesicles are typically assumed to be equivalent. Interestingly, peptide-induced rupture of cholesterol-containing bilayers, formed by adsorbed vesicles yielded supported lipid bilayers that were intractable to MβCD treatment. This evidence suggests one of two possibilities. First, the vesicles may not have contained cholesterol due to mixing heterogeneities, however, the complete absence of cholesterol is unlikely and also the vesicles did not rupture spontaneously on silicon oxide. Second, the peptide may not be completely removed from the bilayer or somehow induce cholesterol in the bilayer to assume a highly ordered, functionally inactive state.

### Example 9

### Formation of Phosphatidylinositol Supported Membranes for Enzyme Monitoring

The SALB method was used to rapidly assemble biomembranes for characterizing membrane-associated drug targets and inhibitor screening. A key example is phosphatidylinositol-related enzymes. Typically, these enzymes are very hard to study because phosphatidylinositols are difficult to incorporate in lipid bilayers. The method was used to reconstitute phosphatidylinositol-related enzymes in a solid supported bilayer (SLB) as a platform. A QCM-D based assay was developed in which SLB containing phosphoinositol (PI) served as a substrate for the kinase and the enzymatic activity was characterized by in situ antibody binding.

### Formation of Supported Lipid Bilayers Containing Phosphatidylinositol 4-Phosphate (PI4P) and Phosphatidylinositol 4,5-Biphosphate [PI(4,5)P2] Using the Method

SLB containing PI was prepared by the solvent-assisted lipid bilayer (SALB) method and used as a substrate for PI4KIIIα and β.

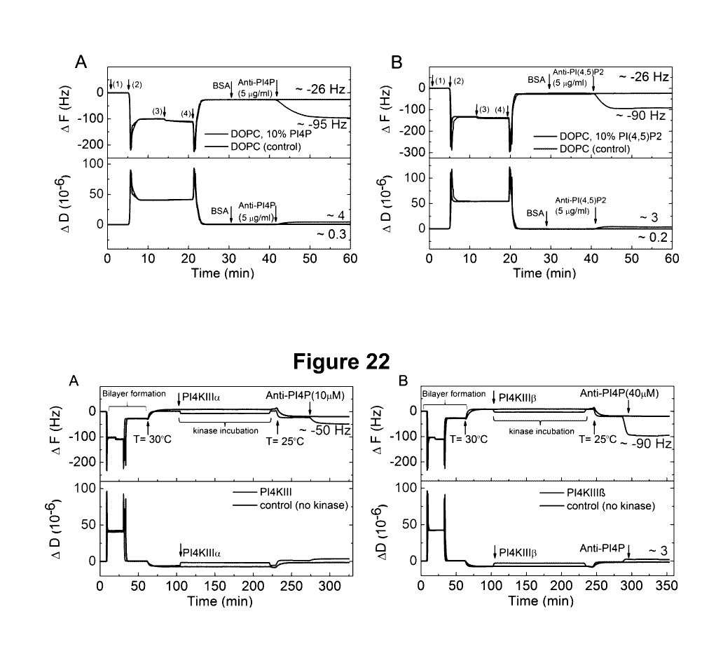

A typical SALB experiment included the following steps (FIG. 21): first, aqueous buffer (10 mM Tris, 150 mM NaCl, pH 7.5) was injected into the QCM-D measurement chamber in order to establish the baseline for the frequency and energy dissipation signals (arrow 1). After 5 min stabilization, the organic solution (i.e., isopropanol) was injected (arrow 2), which leads to a dramatic shift in the baseline within a short transient period. During this stage, the solution does not contain lipid and the observed large shift in the baseline is solely due to the density and viscosity difference between isopropanol and the aqueous buffer. After 10 min additional stabilization, an isopropanol solution containing DOPC and PI4P (10%) [grey curve in FIG. 21A] or pure DOPC [black curve in FIG. 1A] lipid was injected (arrow 3), which led to a decrease in the frequency due to adsorption of lipid at the surface. No changes in the energy dissipation were observed during this stage. At the final stage (arrow 4), the lipid solution (isopropanol+lipid) was replaced by aqueous buffer solution (no lipid). Once the SALB procedure was finished (t=25 min), the Δf and ΔD values were consistent with a planar bilayer (Δf and ΔD shifts of −26 Hz and 0.3×10⁻⁶, respectively). To evaluate the homogeneity of SALB-formed bilayers, the adsorption of proteins at SLB's was examined. Planar bilayers are highly resistant to adsorption of proteins such as e.g., bovine serum albumin (BSA). By contrast, nonspecific protein adsorption on silicon dioxide is generally appreciable. Hence, in principle, protein adsorption on a defect-free bilayer would be negligible and an increasing amount of bound protein suggests the presence of defects, e.g., availability of hydrophilic substrate sites for protein attachment. BSA (0.05 mg/ml) adsorptions on both bilayers resulted in a frequency shift of <−1 Hz, which is negligible. Finally to verify successful incorporation of PI4P, anti-PI4P (40 μM) antibodies were injected. Anti-PI4P adsorption at the PI4P containing bilayer resulted in a dramatic frequency shift of ˜−70 Hz. In contrast, injection of the same antibody to 100 mol % DOPC bilayer resulted in almost no frequency shift indicating no mass increase on the DOPC bilayer. Therefore, frequency shift upon injection of anti-PI4P to PI4P containing bilayer was exclusively-attributed to the specific binding between anti-PI4P and PI4P in the bilayer.

The same protocol was used to form DOPC bilayer containing P1(4,5)P2 (FIG. 21B). To verify incorporation of the phosphoinositols into the bilayers, specific binding of the corresponding antibodies were examined. Subsequent injection of anti-PI4P and anti-PI(4,5)P2 (5 μg/ml) antibodies to the bilayer containing PI4P and PI(4,5)P2 led to further decrease of around 70 Hz in Δf. In contrast, upon injection of antibodies no change in the Δf of pure DOPC bilayers were observed, indicating specific binding of antibodies to the phosphoinositols.

### In Situ Detection of PI4P Generated by Lipid Kinase

Kinase activity was determined by treating PI containing bilayer with PI4KIIIα and β enzymes. Briefly, after bilayer formation the temperature was raised to 30° C. as the maximum kinase activity has been reported at this temperature. Next the buffer was exchanged to kinase buffer [20 mM Tris (pH 7.5), 5 mM MgCl₂, 2 mM DTT, 0.5 mM EGTA, 100 μM ATP] followed by injection of kinase (5 μg/ml). Almost 1 ml of enzyme solution was injected in the course of around 2 hours (FIG. 22). Next the buffer was exchanged to the original buffer and the temperature was set to 23° C. (the temperature at which the bilayer was formed). To detect PI4P generation, anti-PI4P (5 μg/ml) was injected. Antibody binding to bilayers treated with PI4KIIIα and β led to a decrease in Δf until it reached a final value of −50 Hz and −90 Hz respectively. A control experiment was also done in which no enzyme included in the kinase buffer. In that case no antibody binding was observed indicating the antibody binding was specific. Comparing the extent of decrease in the Δf reveals that more PI4P were generated when PI4KIII-β was used, thus this isoform is more active than PI4KIII-α.

### Effects of PI4K Inhibitors on Generation of PI4P

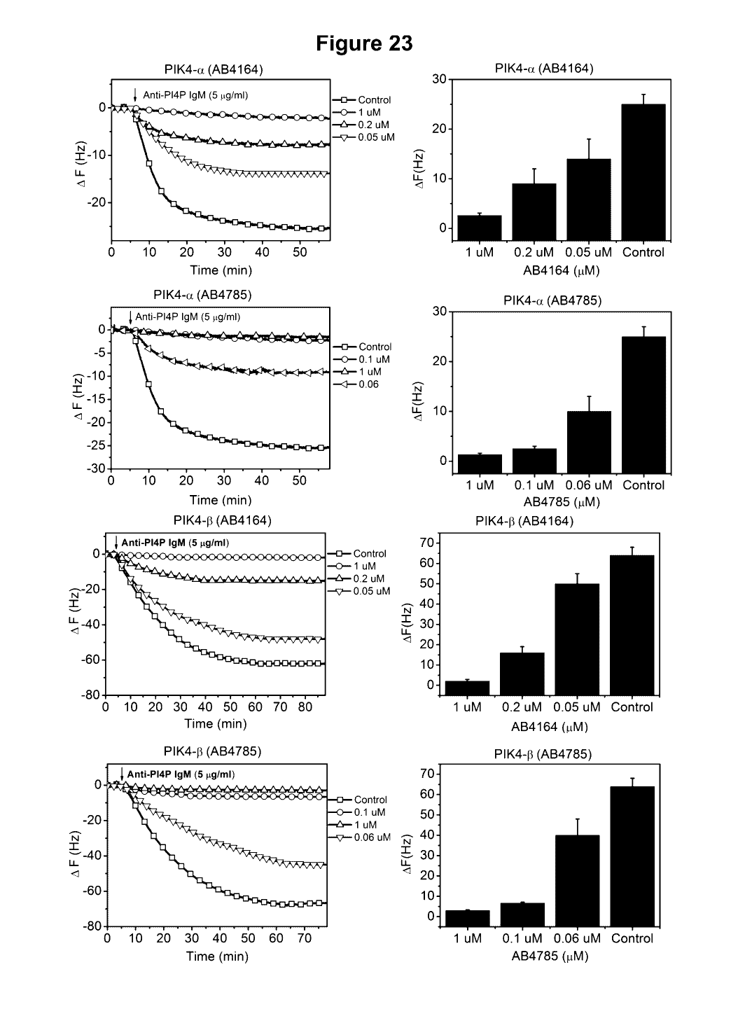

After establishment of the assay, the effect of inhibitors on lipid kinase mediated generation of PI4P was determined. The same experimental procedure and condition as before was repeated but in this case the enzymes were first pre-incubated with inhibitors for 30 min at room temperature. The QCM-D frequency shift was then normalized with ΔFrequency=0 Hz corresponding to the supported, lipid bilayer in each experiment. A solution of 5 μg/ml antibody was next added [see arrow in FIG. 23(A, C, E, and G)] in order to detect the generation of PI4P. The binding curves monitored by changes in Δf are shown in FIG. 23(A, C, E, and G). For each isoform, two inhibitor compounds (AB 4164 and AB 4785) were tested at varying concentrations. The kinetics of antibody bindings shows significant reduction in the presence of inhibitors. The change in Δf upon antibody binding are summarized in FIG. 23(B, D, F, and H). Antibody binding in case of PI4KIIIα and β in the absence of inhibitors led to a decrease in Δf of 25±2 and 64±4 Hz respectively. In the presence of inhibitors in all cases, the decrease in the Δf were below or around 5 Hz.

1. A method for forming a hydration layer/lipid bilayer structure on a solid support comprising:
contacting a solution comprising at least one polar lipid and a water-miscible alcohol as a solvent with the solid support; and
adding water to said solution at a predetermined rate, thus inducing formation of a hydration layer on the solid support surface and formation of a planar lipid bilayer on the hydration layer, wherein the hydration layer has an average thickness of at least 2 nm.

2. The method of claim 1, wherein the hydration layer has a thickness between 2 nm and 4 nm.

3. The method of claim 1 or 2, wherein the solid support has a Hamaker constant in water of at least 3×10⁻²⁰ J.

4. The method of any one of claims 1 to 3, wherein the solid support is selected from the group consisting of silver, gold, aluminum oxide, barium titanate, beryllium oxide, diamond, cadmium sulfide, copper, magnesium aluminate, magnesium oxide, lead sulfide, silicon carbide, silicon nitride, strontium titanate, titanium dioxide, yttrium oxide, zinc sulfide, and zirconium oxide.

5. The method of any one of claims 1 to 4, wherein the solution comprises the polar lipid at a concentration of 0.1 to 0.75 mg/ml, preferably 0.1 to 0.5 mg/ml.

6. The method of any one of claims 1 to 5, wherein the at least one polar lipid comprises at least two different polar lipids.

7. The method of any one of claims 1 to 6, wherein the at least one polar lipid is selected from the group consisting of phospholipids, sphingolipids, fatty acids, derivatives thereof, and combinations thereof.

8. The method of claim 7, wherein the at least one polar lipid comprises at least one phosphoglyceride, preferably a phosphatidylcholine.

9. The method of any one of claims 1 to 8, wherein the solution further comprises another lipid or lipid-like component selected from the group consisting of triacylglycerides and isoprenoids, preferably sterols, more preferably cholesterol.

10. The method of claim 9, wherein the solution comprises up to 60% by weight sterols, preferably cholesterol, relative to the total lipid content.

11. The method of any one of claims 1 to 10, wherein the solution further comprises peptides or proteins that can associate with or insert into the formed lipid bilayer.

12. The method of any one of claims 1 to 11, wherein the alcohol is selected from isopropanol, n-propanol and methanol.

13. The method of any one of claims 1 to 12, wherein the alcohol comprises isopropanol.

14. The method of any one of claims 1 to 13, wherein the solution is essentially non-aqueous.

15. The method of claim 14, wherein the water content of the solution before the step of adding the water is below 40% by weight, preferably below 25% by weight, more preferably below 10% by weight, most preferably below 2% by weight.

16. The method of any one of claims 1 to 15, wherein the water is added in form of an aqueous solution, preferably a buffered aqueous solution.

17. The method of claim 16, wherein the pH of the aqueous solution is in the range of between 7.0 and 7.8, preferably about 7.4.

18. Solid support comprising a hydration layer/lipid bilayer structure, wherein the hydration layer has an average thickness of at least 2 nm and wherein said solid support is obtainable according to the method of any one of claims 1 to 17.

19. The solid support of claim 18, wherein the hydration layer has a thickness between 2 nm and 4 nm.

20. The solid support of claim 18 or 19, wherein the solid support comprises a material having a Hamaker constant in water of at least 3×10⁻²⁰ J.

21. The solid support of any one of claims 18 to 20, wherein the solid support is selected from the group consisting of silver, gold, aluminum oxide, barium titanate, beryllium oxide, diamond, cadmium sulfide, copper, magnesium aluminate, magnesium oxide, lead sulfide, silicon carbide, silicon nitride, strontium titanate, titanium dioxide, yttrium oxide, zinc sulfide, and zirconium oxide.

22. The solid support of any one of claims 18 to 21, wherein the lipid bilayer comprises at least one polar lipid selected from the group consisting of phospholipids, sphingolipids, fatty acids, derivatives thereof, and combinations thereof.

23. The solid support of claim 22, wherein the at least one polar lipid comprises at least one phosphoglyceride, preferably a phosphatidylcholine.

24. The solid support of any one of claims 18 to 23, wherein the lipid bilayer comprises another lipid or lipid-like component selected from the group consisting of triacylglycerides and isoprenoids, preferably sterols, more preferably cholesterol.

25. The solid support of claim 24, wherein the lipid bilayer comprises at least 60 mol % sterols, preferably cholesterol.

26. The solid support of any one of claims 18 to 25, wherein the lipid bilayer further comprises peptides or proteins that can associate with or insert into the formed lipid bilayer.
